(19) United States
(12) Patent Application Publication
(10) Pub. No.: US 2008/0027088 A1
(43) Pub. Date: Jan. 31, 2008

(54) Imidazole-Based Hmg-Coa Reductase Inhibitors

(75) Inventors: Reynold Homan, Ann Arbor, MI (US); Sotirios K. Karathanasis, Carmel, IN (US); Robert L. Panek, Ann Arbor, MI (US); Tae-Sik Park, Ann Arbor, MI (US); Mark D. Rekhter, Carmel, IN (US)

Correspondence Address: PFIZER INC., PATENT DEPARTMENT, MS8260-1611, EASTERN POINT ROAD, GROTON, CT, 06340, US

(73) Assignee: Warner-Lambert Company, LLC

(21) Appl. No.: 10/594,348

(22) PCT Filed: Mar. 21, 2005
(86) PCT No.: PCT/IB05/00733

§ 371 Date: Aug. 15, 2007

Related U.S. Application Data
(60) Provisional application No. 60/557,021, filed on Mar. 26, 2004.

(51) Int. Cl. A61K 31/201; A61K 31/351; A61K 31/405; A61K 31/455; A61K 31/47; A61K 31/505; A61P 3/04; A61P 3/10

(52) U.S. Cl. 514/275; 514/311; 514/356; 514/415; 514/460; 514/560; 514/789

(57) ABSTRACT

The present invention relates to methods of treating atherosclerosis, dyslipidemia, other cardiovascular diseases and related diseases, such as diabetes, using a serine palmitoyltransferase (SPT) inhibitor. The present invention also relates to pharmaceutical compositions and kits that comprise a serine palmitoyltransferase (SPT) inhibitor, optionally with another pharmaceutical agent.

### FIELD OF THE INVENTION

The present invention relates to methods of using a compound that is a serine palmitoyltransferase (SPT) inhibitor to elevate certain plasma lipid levels, including high density lipoprotein (HDL)-cholesterol, and to lower other plasma lipid levels such as low density lipoprotein (LDL)-cholesterol and triglycerides, and accordingly to treat diseases which are affected by low levels of HDL cholesterol and/or high levels of LDL-cholesterol and triglycerides, such as atherosclerosis, dyslipidemia, hypercholesterolemia, hypertriglyceridemia, cardiovascular diseases and related diseases such as diabetes. The present invention also relates to pharmaceutical compositions and kits that comprise a SPT inhibitor and a second therapeutic agent.

### BACKGROUND OF THE INVENTION

Atherosclerosis and its associated coronary artery disease (CAD) is the leading cause of mortality in the industrialized world. Despite attempts to modify secondary risk factors (e.g., smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

The pathological sequence leading to atherosclerosis and coronary heart disease is well known. The earliest stage in this sequence is the formation of “fatty streaks” in the carotid, coronary and cerebral arteries and in the aorta. These lesions are yellow in color due to the presence of lipid deposits found principally within smooth-muscle cells and in macrophages of the intima layer of the arteries and aorta. Further, it is postulated that most of the cholesterol found within the fatty streaks, in turn, give rise to development of “fibrous plaques,” which consist of accumulated intimal smooth muscle cells laden with lipid and are surrounded by extra-cellular lipid, collagen, elastin and proteoglycans. The cells plus matrix form a fibrous cap that covers a deeper deposit of cell debris and more extra-cellular lipid. The lipid is primarily free and esterified cholesterol. The fibrous plaque forms slowly, and is likely in time to become calcified and necrotic, advancing to a “complicated lesion,” which accounts for arterial occlusion and tendency toward mural thrombosis and arterial muscle spasm that characterize advanced atherosclerosis.

Risk for development of atherosclerosis and related cardiovascular disease has been shown to be strongly correlated with certain plasma lipid levels. In recent years, leaders of the medical profession have placed renewed emphasis on lowering plasma cholesterol levels, and low density lipoprotein (LDL)-cholesterol, in particular. The upper limits of “normal” are now known to be significantly lower than heretofore appreciated. As a result, large segments of Western populations are now realized to be at particularly high risk. Such independent risk factors include glucose intolerance, left ventricular hypertrophy, hypertension, and being of the male sex. Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance.

While elevated LDL-cholesterol may be the most recognized form of dyslipidemia, it is by no means the only significant lipid associated contributor to CHD. Low HDL-C is also a known risk factor for CHD (D. J. Gordon et al., “High-density Lipoprotein Cholesterol and Cardiovascular Disease,” Circulation (1989) 79: 8-15). High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

No wholly satisfactory lipid-modulating therapies exist. Niacin can significantly increase HDL-cholesterol, but has serious toleration issues, which reduce compliance. Fibrates and the HMG-CoA reductase inhibitors lower LDL-cholesterol but raise HDL-cholesterol only modestly (˜10-12%). As a result, there is a significant unmet medical need for a well-tolerated agent, which can lower plasma LDL levels and/or elevate plasma HDL levels (i.e., improving the patient's plasma lipid profile), thereby reversing or slowing the progression of atherosclerosis.

Thus, although there are a variety of anti-atherosclerosis therapies, there is a continuing need and a continuing search for alternative therapies for the treatment of atherosclerosis and dyslipidemia.

Serine palmitoyltransferase (SPT) catalyzes the first committed step in sphingolipid synthesis (FIG. 1). SPT condenses the palmitic acid of palmitoyl-coenzyme A with serine to produce ketosphinganine, the initial precursor to the unique aminolipid backbone that is characteristic of all sphingolipids (K. Hanada et al., J. Biol.Chem. 1997;272(51):32108-14). SPT is composed of two different subunits, LCB1 and LCB2 (B. Weiss and W. Stoffel, Eur.J.Biochem. 1997;249(1):239-47; see also WO 99/49021.) LCB1 and LCB2 genes are essential for cell survival and the changes in SPT activity result in a defective development of the fruit fly and filamentous fungi (J. Cheng et al., Mol. Cell. Biol. 2001 ;21 (18):6198-209; and T. Adachi-Yamada et al., Mol. Cell. Biol. 1999;19(10):7276-86), and hereditary sensory neuropathy type I in humans (J.L. Dawkins et al., Nat. Genet. 2001;27; (3):309-12; and K. Bejaoui et al., Nat. Genet. 2001 ;27(3):261-2).

Sphingomyelin is one of the major phospholipids in plasma lipoproteins and cell membranes. In vitro studies have demonstrated that sphingomyelin and related sphingolipids are proatherogenic in a variety of circumstances and have identified a positive correlation between plasma sphingomyelin (SM) content and the incidence of coronary artery disease (X. Jiang et al., Arterioscler.Thromb.Vasc.Biol. 2000; 20:2614-2618; and R.D. Williams, et al., J. Lipid Res. 1986. 27:763-770). SM and its derivatives are accumulated in human and experimental atherosclerotic lesions (S. L. Schissel et al., J Clin Invest. 1996;98(6):1455-64). Intermediates of SM synthesis, in particular, ceramide, also possess independent pro-atherogenic properties. Ceramide plays an important role in lipoprotein aggregation and may promote foam cell formation (K. J. Williams and I. Tabas, Arterioscler. Thromb. Vasc. Biol. 1995;15:551-561).

Although direct mechanistic links between SM and atherosclerosis have not been established, available in vitro data suggests that SM might have the following proatherogenic properties. First, increased SM content of HDL and triglyceride-rich lipoproteins, for example, is shown to obstruct reverse cholesterol transport and trigylceride-rich lipoprotein clearance by interfering with the activities of lecithin:cholesterol acyltransferase (LCAT) (D.J. Bolin and A. Jonas, J. Biol.Chem. 1996;271(32):19152-8) and lipoprotein lipase (LPL) (I. Arimoto et al., J. Lipid Res. 1998;39(1):143-51; I. Arimoto et al., Lipids 33:773-779 (1996); and H. Saito et al., Biochimica et Biophysica Acta 1486 (2000) 312-320), respectively. It has also been demonstrated that SM in macrophage membranes interfered with reverse cholesterol transport (A. R. Leventhal et al., J. Biol. Chem. 2001;276(48):44976-83).

Second, SM-rich lipoproteins can be converted to foam cell substrates by sphingomyelinase in the artery wall (S. L. Schissel et al., J. Biol. Chem. 1998;273(5):2738-46), thereby promoting foam cell formation.

Third, ceramide and related products of SM synthesis and breakdown are potent regulators of cell proliferation, activation and apoptosis (M. Maceyka et al., Biochim. Biophys. Acta. 2002;1585(2-3):193-201) and hence may affect plaque growth and stability.

Other proatherogenic effects of sphingolipids include the observation that SM in LDL enhances the reactivity of LDL with sphingomyelinase, which is released by macrophages in the artery wall (Ts. Jeong et al., J. Clin. Invest. 1998;101 (4):905-912). This process results in LDL aggregation and subsequent foam cell formation (S. L. Schissel et al., J. Clin. Invest. 1996;98(6):1455-1464). Increased sphingomyelin content in plasma membranes is also known to reduce reverse cholesterol transport by impeding the transfer of cellular cholesterol to HDL (R. Kronqvist et al., Eur. J. Biochem. 1999;262:939-946). Furthermore, SPT activation is strongly implicated in Fas-mediated apoptosis, which could promote plaque destabilization. Fas activation causes apoptosis in macrophages (P. M. Yao and I. Tabas, J. Biol. Chem. 2000;275:23807-23813) and smooth muscle cells (A. C. Knapp et al., Athero. 2000;152:217-227). Fas activation depends on de novo synthesis of ceramide, a product of SPT and an SM precursor (A. Cremesti et al., J. Biol. Chem. 2001;276:23954-23961).

Genes regulating cholesterol synthesis contain sterol regulatory elements (SREs) in their promoter regions (J. D. Horton, J. L. Goldstein and M. S. Brown, J. Clin. Invest. 2002;109(9):1125-31). Through several intermediate steps, SREs are controlled by intra-cellular free cholesterol (M. S. Brown and J. L. Goldstein, Cell. 1997;89(3):331-40). SM, a major plasma membrane component, has a high affinity for free cholesterol (T. S. Worgall et al., J. Biol. Chem. 200;277(6):3878-85; and V. Puri et al., J. Biol. Chem. 2003;278(23):20961-70). It has been reported that SM depletion by sphingomyelinase treatment causes an increased cholesterol translocation to endoplastic reticulum and suppression of SREBP cleavage (S. Sheek, M. S. Brown and J. L. Goldstein, Proc. NatI. Acad. Sci. U.S.A. 1997;94(21):11179-83). Recent findings demonstrated that inhibition of sphingolipid biosynthesis caused suppression of lipogenic gene expression in Chinese hamster ovary cells (T. S. Worgall et al., Arterioscler. Thromb. Vasc. Biol. 2004; 24: 943-948).

SPT inhibitors are known to block ceramide production and the resultant apoptosis in cardiomyocytes (D. Dyntar et al., Diabetes 2001;50:2105-2113) and the insulin-producing pancreatic β-cells (M. Shimabukuro et al., Proc. Nat. Acad. Sci. 1998;95(5):2498-2502). SPT inhibition prevents apoptosis of islets of prediabetic fa/fa rats (M. Shimabukuro et al., J. Biol. Chem. 1998;273(49):32487-90). Recent findings also demonstrated that palmitate inhibits preproinsulin gene expression via ceramide biosynthesis. SPT inhibition recovered expression of preproinsulin in rat islet culture and improved the insulin production (C. L. Kelpe et al., J. Biol. Chem. 2003;278(32):30015-21).

Myriocin is a known serine palmitoyltransferase (SPT) inhibitor (K. Hanada et al., Biochem.Pharmacol. 2000;59:1211-1216; and J. K. Chen et al., Chemistry & Biology 1999;6:221-235) isolated from fungi (Y. Miyake et al., Biochem. Biophys. Res. Commun. 1995;211 (2):396-403), which is commercially available, and known to have a potent immunosuppressive activity (T. Fujita et al., J. Antibiot. (Tokyo) 1994;47(2):208-15). It has been shown that myriocin possesses immunomodulatory properties independent of its ability to inhibit SPT and via growth inhibition in T-lymphocytes.

WO 01/80903 discloses detection and treatment of atherosclerosis based on plasma sphingomyelin concentration.

WO 02/074924 and U.S. 2002/0197654, Thromb. Haemost., 2001;86:1320-1326; disclose a method for comparatively measuring the level of normal and hyperproliferative serine palmitoyltransferase expression in a mammalian cell and uses thereof, such as detecting cancer or treating restenosis.

U.S. 2003/9996022 discloses methods and compositions useful for treating or preventing cardiovascular or cerebrovascular disease through the use of agents that interfere with the production and/or biological activities of sphingolipids and their metabolites, particularly sphingosine (SPH) and sphingosine1-phosphate (S-1-P).

WO 01/80715 discloses methods for identifying compounds useful for preventing acute clinical vascular events in a subject.

U.S. Pat. No. 6,613,322; US2003/0026796 and WO 99/11283 disclose methods for treating a subject suffering from an atherosclerotic vascular disease comprising administering to the subject an amount of a zinc sphingomyelinase inhibitor effective to decrease extracellular zinc sphingomyelinase activity in the subject.

Tae-Sik Park et al., Circulation. 2004;110:3465-3471, describes the reduction of atherogenesis in Apo-E knockout mice by the inhibition of sphingomyelin synthesis.

M. Hojjati et al., JBC Papers in Press, Published on Dec. 6, 2004, as Manuscript M412348200,describes the effect of myriocin on plasma sphingolipid metabolism and atherosclerosis in apoE-deficient mice.

### SUMMARY OF THE INVENTION

The present invention provides the following therapeutic methods: methods of lowering plasma lipids comprising administering a therapeutically effective amount of a serine palmitoyltransferase (SPT) inhibitor to a mammal in need thereof; methods for elevating high density lipoprotein (HDL) particles comprising administering a therapeutically effective amount of a serine palmitoyltransferase (SPT) inhibitor to a mammal in need thereof; methods for lowering very low density lipoprotein (VLDL) particles and low density lipoprotein (LDL) particles comprising administering a therapeutically effective amount of a serine palmitoyltransferase (SPT) inhibitor to a mammal in need thereof; methods for lowering plasma triglyercides particles comprising administering a therapeutically effective amount of a serine palmitoyltransferase (SPT) inhibitor to a mammal in need thereof; methods for lowering serum levels of total cholesterol comprising administering a therapeutically effective amount of a serine palmitoyltransferase (SPT) inhibitor to a mammal in need thereof; methods for improving plasma lipid profile comprising administering a therapeutically effective amount of a serine palmitoyltransferase (SPT) inhibitor to a mammal in need thereof; methods for inhibiting plaque formation comprising administering a therapeutically effective amount of a serine palmitoyltransferase (SPT) inhibitor to a mammal in need thereof; methods of reducing the size of plaque comprising administering a therapeutically effective amount of a serine palmitoyltransferase (SPT) inhibitor to a mammal in need thereof; methods of reducing the size of an atherosclerotic lesion comprising administering a therapeutically effective amount of a serine palmitoyltransferase (SPT) inhibitor to a mammal in need thereof; methods of reducing the size of a macrophage foam cell comprising administering a therapeutically effective amount of a serine palmitoyltransferase (SPT) inhibitor to a mammal in need thereof; methods for preventing plaque rupture comprising administering a therapeutically effective amount of a serine palmitoyltransferase (SPT) inhibitor to a mammal in need thereof; methods for treating dyslipidemia which comprise administering a therapeutically effective amount of a serine palmitoyltransferase (SPT) inhibitor to a mammal in need thereof; methods for treating atherosclerosis which comprise administering a therapeutically effective amount of a serine palmitoyltransferase (SPT) inhibitor to a mammal in need thereof; methods for treating diabetes which comprise administering a therapeutically effective amount of a serine palmitoyltransferase (SPT) inhibitor to a mammal in need thereof; methods for treating metabolic syndrome which comprise administering a therapeutically effective amount of a serine palmitoyltransferase (SPT) inhibitor to a mammal in need thereof; and finally, methods for treating inflammation which cornprise administering a therapeutically effective amount of a serine palmitoyltransferase (SPT) inhibitor to a mammal in need thereof. More particularly, the present invention provides such methods in which the SPT inhibitor is myriocin.

In addition, the present invention provides pharmaceutical compositions comprising: a) a compound that is a serine palmitoyltransferase (SPT) inhibitor; and b) a second compound useful for the treatment of atherosclerosis or dyslipidemia. More particularly, the present invention provides such compositions wherein the second compound is an HMG-CoA reductase inhibitor, an HMG-CoA synthase inhibitor, an HMG-CoA reductase gene expression inhibitor, an HMG-CoA synthase gene expression inhibitor, a CETP inhibitor, a bile acid sequestrant, a cholesterol absorption inhibitor, a cholesterol biosynthesis inhibitor, a squalene synthetase inhibitor, a fibrate, niacin, a combination of niacin and lovastatin and an antioxidant. Even more particularly, the present invention provides such compositions wherein the second compound is an HMG-CoA reductase inhibitor. Most particularly, the present invention provides such compositions wherein the second compound is lovastatin, simvastatin, pravastatin, fluvastatin, atorvastatin, rivastatin, rosuvastatin or pitavastatin. The present invention also provides such compositions wherein the second compound is a CETP inhibitor. More particularly, the present invention provides such compositions wherein the second compound is torcetrapib. The present invention also provides such compositions wherein the SPT inhibitor is myriocin.

Also, the present invention provides kits that comprises: a) a serine palmitoyltransferase (SPT) inhibitor and a pharmaceutically acceptable carrier, vehicle or diluent in a first unit dosage form; b) a second compound that is useful for the treatment of atherosclerosis or dyslipidemia and a pharmaceutically acceptable carrier, vehicle or diluent in a second unit dosage form; and c) a means for containing the first and second unit dosage forms. More particularly, the present invention provides such kits wherein the second compound is an HMG-CoA reductase inhibitor, an HMG-CoA synthase inhibitor, an HMG-CoA reductase gene expression inhibitor, an HMG-CoA synthase gene expression inhibitor, a CETP inhibitor, a bile acid sequestrant, a cholesterol absorption inhibitor, a cholesterol biosynthesis inhibitor, a squalene synthetase inhibitor, a fibrate, niacin, a combination of niacin and lovastatin and an antioxidant; and a pharmaceutically acceptable carrier, vehicle or diluent in a second unit dosage form; wherein the amounts of first and second compounds result in a therapeutic effect. Even more particularly, the present invention provides such kits wherein the second compound is an HMG-CoA reductase inhibitor. Most particularly, the present invention provides such kits wherein the second compound is lovastatin, simvastatin, pravastatin, fluvastatin, atorvastatin, rivastatin, rosuvastatin or pitavastatin. In addition, the present invention provides such kits wherein the second compound is a CETP inhibitor. More particularly, the present invention provides such kits wherein the second compound is torcetrapib. Also, the present invention provides such kits wherein the SPT inhibitor is myriocin.

The present invention also provides the use of a serine palmitoyltransferase (SPT) inhibitor for the manufacture or preparation of a medicament for the treatment of a mammal in need thereof, as described above.

As noted above, in clinical studies, sphingomyelin (SM) plasma levels have been correlated with the occurrence of coronary heart disease, independently of plasma cholesterol levels. Myriocin is a potent inhibitor of serine palmitoyltransferase (SPT), the rate-limiting enzyme in ceramide and sphingomyelin (SM) biosynthesis. In the present invention, it has been found that inhibition of de novo SM biosynthesis, using myriocin, improves the lipid profile and reduces atherogenesis in the ApoE knockout (KO) mouse. Therefore, the present invention is directed to the uses of SPT inhibitors for treating atherosclerosis, dyslipidemia and related diseases.

### BRIEF DESCRIPTION OF THE FIGURES

The present invention is further described by the following nonlimiting examples, which refer to the accompanying FIGS. 1-14, short particulars of which are given below.

In the present invention, SPT inhibition has been further assessed by measuring plasma and tissue sphingomyelin, ceramide or sphinganine as a biomarker for inhibition. The present studies relate to an effect of a specific and commercially available SPT inhibitor, myriocin, on lipid-lowering and the prevention of atherosclerosis in the ApoE knockout (KO) mouse, an atherosclerosis-prone model.

FIG. 1. Sphingomyelin Biosynthetic Pathway.

Serine palmitoyltransferase (SPT) is the first rate-limiting step of sphingolipid biosynthesis. Myriocin specifically inhibits the SPT reaction.

FIGS. 1-A, 1-B, 1-C, 1-D and 1-E. SPT gene expression and enzyme activity.

FIG. 2. Effect of myriocin on the plasma lipoprotein distribution of ApoE KO mice fed a Western diet for 4 weeks. HDL-high density lipoprotein (FIG. 2-C); LDL-low density lipoprotein (FIG. 2-B); VLDL-very low density lipoprotein (FIG. 2-A).

The SPT inhibitor, myriocin, was administered to Western diet-fed ApoE KO mice as diet admix for 4 weeks at doses of 0 (control), 0.1, 0.3, and 1.0 mg/kg/day. Myriocin caused a dose-dependent elevation of HDL-C, and lowered apoB-containing lipoproteins, LDL and VLDL (FIG. 2).

FIG. 3. Total cholesterol and triglyceride concentrations in the plasma of ApoE KO mice fed a Western diet for 4 weeks in the presence of myriocin..

The control ApoE KO mice were fed only Western diet. Total plasma cholesterol (FIG. 3A) and triglycerides (FIG. 3B) were also reduced by myriocin.

FIG. 4. Effect of myriocin on the plasma and liver Sphingomyelin in ApoE KO mice fed a Western diet for 4 weeks.

Sphingomyelin was analyzed by LC/MS. In addition, plasma and liver sphingomyelin concentrations (a potential mechanism-based biomarker) were reduced in a dose-dependent fashion (FIG. 4).

FIG. 5. Effect of myriocin on lesion development in the cuffed femoral artery of ApoE KO mice fed a Western diet for 4 weeks.

The lipid profile changes were accompanied by a significant reduction of atherosclerotic lesions in the femoral artery cuff model (Arteriosclerosis and Thrombosis, Vol. 13, 1874-1884,1993) (FIG. 5A). Plasma serum amyloid A levels were also determined (FIG. 5B). Atherosclerotic lesion (black bars) and macrophage size (gray bars) in the femoral artery were quantified.

FIG. 6. Effect of myriocin on the plasma lipoprotein distribution of ApoE KO mice fed a Western diet for 12 weeks.

In a single dose study, the SPT inhibitor, myriocin, (0.3 mg/kg) was administered to Western diet-fed ApoE KO mice as diet admix for 12 weeks. Cholesterol profile in lipoproteins was examined using isolated plasma by FPLC. Myriocin treatment lowered the VLDL- and LDL-cholesterol and increased HDL, respectively (FIGS. 6A, B and C) when compared with Western diet-fed ApoE KO mice. Cholesterol levels in lipoproteins of myriocin-treated ApoE KO mice were comparable to normal chow-fed ApoE KO mice. C57BI/6J control mice showed very low total cholesterol in plasma.

FIG. 7. Effect of myriocin (0.3 mg/kg, diet ad-mix) on the total cholesterol and triglyceride concentrations of ApoE KO mice fed a Western diet for 12 weeks.

Total cholesterol levels in plasma of Western diet plus myriocin-fed ApoE KO mice was lowered when compared to Western diet-fed group (FIG. 7A). In addition, rnyriocin treatment lowered the plasma triglyceride levels (FIG. 7B). Total cholesterol and triglycerides levels in plasma of myriocin-treated ApoE KO mice were comparable to normal chow-fed ApoE KO mice. On the other hand, the wild type C57BI/6J mice showed low level of plasma total cholesterol and of triglyceride levels.

FIG. 8. Effect of myriocin (0.3 mg/kg, diet ad-mix) on liver, plasma and aorta sphingomyelin (SM) concentrations in ApoE KO mice fed a Western diet for 12 weeks.

Myriocin treatment lowered SM accumulation in the liver (FIG. 8A). The Western diet-fed ApoE KO mice displayed the highest level of plasma SM. Myriocin treatment lowered plasma SM in Western diet-fed ApoE KO mice (FIG. 8B). Small differences were observed among aortas of various treatments. However, there were statistically significant differences between Western diet-fed ApoE KO and C57BI/6J control mice; myriocin decreased SM levels in the aorta (FIG. 8C)

FIG. 9. Effect of myriocin (0.3 mg/kg, diet ad-mix) on liver and aorta sphinganine concentrations in ApoE KO mice fed a Western diet for 12 weeks.

Sphinganine levels were significantly increased in Western diet-fed as well as normal chow-fed ApoE KO mice compared to control C57BI/6J mice. Myriocin treatrnent lowered sphinganine levels in liver when compared to the Western diet-fed ApoE KO mice (FIG. 9A). In aorta, sphinganine levels in the myriocin-treated ApoE KO mice, normal chow-fed ApoE KO mice, and C57BI/6J control mice were lower than the Western diet-fed ApoE KO mice (FIG. 9B)

FIG. 10. Effect of myriocin on lipid deposition in aortae of Western-diet fed ApoE KO mice.

Oil Red O staining of en face aortas revealed that myriocin treatment reduced atherosclerotic lesion coverage in Western diet-fed ApoE KO mice (FIG. 10).

FIG. 11. Effect of myriocin (0.3 mg/kg, diet ad-mix) on formation of total lesion and macrophage area in the aortic root.

FIG. 12. Effect of myriocin (0.3mg/kg, diet ad-mix) on formation of total lesion and macrophage area in the brachiocephalic artery.

ApoE KO mice were fed Western-diet in the absence or presence of myriocin. ApoE KO mice and C57BI/6J control mice fed with normal chow were sacrificed. Atherosclerotic lesion (black bars) and macrophage size (gray bars) in the cross-sections of aortic root and brachiocephalic artery were quantified by using Image Pro Plus (FIGS. 11-12).

FIG. 13. SM/PC ratio and ceramide concentrations in plasma.

Myriocin treatment reduced ceramide levels and was not associated with any changes in the SM/PC ratio.

FIG. 14. Incorporation of T lymphocytes into lesion of aortic root.

Accumulation of T cells was not affected by myriocin treatment.

### DETAILED DESCRIPTION OF THE INVENTION

The present invention relates to methods of treating atherosclerosis, dyslipidemia, other cardiovascular diseases and related diseases, such as diabetes, using a compound that is a serine palmitoyltransferase (SPT) inhibitor. In addition, the present invention provides pharmaceutical compositions and kits comprising a serine palmitoyltransferase (SPT) inhibitor.

In accordance with the present invention, atherosclerosis, dyslipidemia, other cardiovascular diseases and related diseases, such as diabetes, can be treated by administering to a patient having or at risk of having such diseases a therapeutically effective amount of a serine palmitoyltransferase (SPT) inhibitor.

As shown in the Examples below, it has been demonstrated in the present invention that SM content and production were proportionally increased in plasma, liver and aorta of the Western diet-fed ApoE KO mice compared to standard chow-fed ApoE KO and C57BI/6J control mice. Myriocin, a specific inhibitor of SPT, inhibited de novo SM synthesis in the liver and aorta; this was associated with reductions of plasma SM and ceramide that were not accompanied by changes in SM/PC ratio. Inhibition of SM synthesis led to the lowering of plasma cholesterol and triglyerides. These changes were associated with dramatic anti-atherosclerotic effects in vivo.

SM depletion was also associated with an elevation of HDL. In vitro data suggest that increased SM content in lipoproteins can inhibit key enzymes involved in lipoprotein metabolism. It has also been demonstrated that SM in macrophage membranes interfered with reverse cholesterol transport. It is conceivable that SM depletion would lead to activation of reverse cholesterol transport and contribute to elevation of HDL cholesterol, which is consistent with observations from the present invention.

In the present invention, it has been demonstrated that inhibition of SM synthesis was associated with significant reductions in atherosclerotic lesion formation in ApoE KO mice. Since plaque formation in ApoE KO mice is lipid-driven, the observed anti-atherogenic effects were likely indirect, due to normalization of plasma lipids as a result of the inhibition of SM synthesis by the liver. However, local inhibition of SM production in the aorta has also been shown. Myriocin-treated, Western diet-fed ApoE KO mice showed a plasma lipid profile similar to that in the standard chow-fed ApoE KO mice, but their lesions were significantly smaller. Taken together, these findings suggest that anti-atherogenic effects of myriocin could, in part, be attributed to the local inhibition of SPT in the arterial wall.

Thus, SPT inhibition by myriocin in ApoE KO mice effectively inhibited SM synthesis, an effect that was associated with an improved plasma lipid profile and significant anti-atherogenic activity. Consistent with these observations are clinical reports indicating that SM is an independent risk factor for coronary heart disease and a plasma marker of coronary artery disease. The present invention shows that SPT and potentially other key enzymes regulating SM synthesis could represent a novel class of molecular targets for prevention of dyslipidemia, atherosclerosis and related diseases.

The term “therapeutically effective amount” means an amount of a compound or combination of compounds that treats a disease; ameliorates, attenuates, or eliminates one or more symptoms of a particular disease; or prevents or delays the onset of one of more symptoms of a disease.

The term “patient” means animals, such as dogs, cats, cows, horses, sheep, geese, and humans. Particularly preferred patients are mammals, including humans of both sexes.

The term “pharmaceutically acceptable” means that the substance or composition must be compatible with the other ingredients of a formulation, and not deleterious to the patient.

The terms “treating”, “treat” or “treatment” include preventative (e.g., prophylactic) and palliative treatment.

The term “serine palmitoyltransferase (SPT) inhibitor” means a compound or a pharmaceutically acceptable salt thereof, which inhibits or blocks the enzyme, serine palmitoyltransferase (SPT). It is also contemplated that any additional pharmaceutically active compound used in combination with a serine palmitoyltransferase (SPT) inhibitor can be a pharmaceutically acceptable salt of the additional active compound. The term “SPT inhibitor” includes, for example, synthetic or natural amino acid polypeptides, proteins, small synthetic organic molecules, or deoxy- or ribo-nucleic acid sequences that bind to serine palymitoyltransferase with about 20-fold or greater affinity compared to other proteins or nucleic acids. For example, but not by way of limitation, polyclonal or monoclonal (including classical or phage display) antibodies raised against the serine palmitoyltransferase protein or a peptide fragment thereof or nucleic acid probes that hybridize with serine palmitoyltransferase mRNA are suitable for use in the present invention.

The term “selective” means that a ligand binds with greater affinity to a particular receptor when compared with the binding affinity of the ligand to another receptor. Preferably, the binding affinity of the ligand for the first receptor is about 50% or greater than the binding affinity for the second receptor. More preferably, the binding affinity of the ligand to the first receptor is about 75% or greater than the binding affinity to the second receptor. Most preferably, the binding affinity of the ligand to the first receptor is about 90% or greater than the binding affinity to the second receptor.

Serine palmitoyltransferase (SPT) inhibitors can be identified, for example, by screening a compound library. Methods of identifying inhibitors of enzymes are well known to those skilled in the art. Specific procedures that can be used to identify serine palmitoyltransferase (SPT) inhibitors are presented in other publications, such as WO01/80913; U.S. 2002/0197654; K. Hanada, T. Hara and M. Nishijima, J. Biol. Chem., 24 Mar. 2000; 275(12):8409-15; and K. Gable et al., J. Biol. Chem., 17 Mar. 2000; 275(11):7597-603; which are hereby incorporated by reference herein. Novel inhibitors are discovered using methods that measure serine palmitoyltransferase enzymatic activity.

Examples of known serine palmitoyltransferase (SPT) inhibitors include myriocin, which is commercially available, D-cycloserine, sphingofungin B, sphingofungin C and viridiofungins. Other SPT inhibitors will be known to those skilled in the art, for example, those disclosed in WO 01/80903, such as lipoxamycin and haloalanines (J.K. Chen, Chemistry & Biology, April 1999, Vol. 6:221-235; and U.S. 2002/0197654).

The term “pharmaceutically acceptable salts” includes the salts of compounds that are, within the scope of sound medical judgment, suitable for use with patients without undue toxicity, irritation, allergic response, and the like, commensurate with a reasonable benefit/risk ratio, and effective for their intended use, as well as the zwitterionic forms, where possible, of the compounds.

The term “salts” refers to inorganic and organic salts of compounds. These salts can be prepared in situ during the final isolation and purification of a compound, or by separately reacting a purified compound with a suitable organic or inorganic acid or base, as appropriate, and isolating the salt thus forrned. Representative salts include the hydrobromide, hydrochloride, sulfate, bisulfate, nitrate, acetate, oxalate, palmitiate, stearate, laurate, borate, benzoate, lactate, phosphate, tosylate, besylate, esylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66:1-19 (1977).

A serine palmitoyltransferase (SPT) inhibitor may contain asymmetric or chiral centers, and therefore, exist in different stereoisomeric forms. It is contemplated that all stereoisomeric forrns as well as mixtures thereof, including racemic mixtures, form part of the present invention. In addition, the present invention contemplates all geometric and positional isomers. For example, if a compound contains a double bond, both the cis and trans forms, as well as mixtures, are contemplated.

Mixtures of isomers, including stereoisomers can be separated into their individual isomers on the basis of their physical chemical differences by methods well know to those skilled in the art, such as by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Also, some of the compounds of this invention may be atropisomers (e.g., substituted biaryls) and are considered as part of this invention.

A serine palmitoyltransferase (SPT) inhibitor may exist in unsolvated as well as solvated forms with pharmaceutically acceptable solvents such as water, ethanol, and the like. The present invention contemplates and encompasses both the solvated and unsolvated forms.

It is also possible that a serine palmitoyltransferase (SPT) inhibitor may exist in different tautomeric forms. All tautomers of a serine palmitoyltransferase (SPT) inhibitor are contemplated.

It is also intended that the invention disclosed herein encompass compounds that are synthesized in vitro using laboratory techniques, such as those well known to synthetic chemists; or synthesized using in vivo techniques, such as through metabolism, fermentation, digestion, and the like. It is also contemplated that compounds may be synthesized using a combination of in vitro and in vivo techniques.

The present invention also includes isotopically labeled compounds, which are identical to the non-isotopically labeled compounds, but for the fact that one or more atoms are replaced by an atom having an atomic mass or mass number different from the atomic mass or mass number usually found most abundantly in nature. Examples of isotopes that can be incorporated into compounds identified by the present invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, 35S, ¹⁸F, ¹³⁵ I and ³⁶Cl, respectively. SPT inhibitors and pharmaceutically acceptable salts thereof, which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds can generally be prepared by substituting a readily available isotopically labeled reagent for a non-isotopically labeled reagent.

Metabolic syndrome, also known as Syndrome X or insulin resistance, refers to a common clinical disorder that is defined as the presence of increased insulin concentrations in association with other disorders including viceral obesity, hyperlipidemia, dyslipidemia, hyperglycemia, hypertension, and potentially hyperuricemis and renal dysfunction.

A serine palmitoyltransferase (SPT) inhibitor is administered to a patient in a therapeutically effective amount. A serine palmitoyltransferase (SPT) inhibitor can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using transdermal delivery. It is also noted that the dose of the compound can be varied over time. A serine palmitoyltransferase (SPT) inhibitor can be administered using an immediate release formulation, a controlled release formulation, or combinations thereof. The term “controlled release” includes sustained release, delayed release, and combinations thereof.

A serine palmitoyltransferase (SPT) inhibitor and other pharmaceutically active compounds, if desired, can be administered to a patient orally, rectally, parenterally, (for example, intravenously, intramuscularly, or subcutaneously) intracisternally, intravaginally, intraperitoneally, intravesically, locally (for example, powders, ointments or drops), or as a buccal or nasal spray.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. A preferred carrier is Miglyol®. Proper fluidity can be maintained, for example, by the use of a coating such as lecithin, by the maintenance of the required particle size in the case of dispersions, and/or by the use of surfactants.

These compositions may also contain adjuvants such as preserving, wefting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxym-ethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, Miglyol®, glycerol, tetrahydrofurfuryl alcohol, polyethylene glycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound, may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Compositions for rectal or vaginal administration can be prepared by mixing a serine palmitoyltransferase (SPT) inhibitor and any additional compounds with suitable non-irritating excipients or carriers such as cocoa bufter, polyethylene glycol or a suppository wax, which are solid at ordinary room temperature, but liquid at body temperature, and therefore, melt in the rectum or vaginal cavity and release the compound.

Dosage forms for topical administration of a serine palmitoyltransferase (SPT) inhibitor include ointments, powders, sprays and inhalants. The compound(s) are admixed under sterile conditions with a physiologically acceptable carrier, and any preservatives, buffers, and/or propellants that may be required. Opthalmic formulations, eye ointments, powders, and solutions are also contemplated as being within the scope of this invention.

A serine palmitoyltransferase (SPT) inhibitor can be administered to a patient at dosage levels in the range of about 0.1 to about 7,000 mg per day. A preferred dosage range is about 1 to about 100 mg per day. The specific dosage and dosage range that can be used depends on a number of factors, including the requirements of the patient, the severity of the condition or disease being treated, and the pharmacological activity of the compound being administered. The determination of dosage ranges and optimal dosages for a particular patient is well within the ordinary skill of one in the art in view of this disclosure.

The present invention relates to the use of serine palmitoyltransferase (SPT) inhibitors to treat atherosclerosis, dyslipidemia and other cardiovascular diseases. The methods of treatment of the present invention can also include combination therapy where other pharmaceutically active compounds useful for the treatment of atherosclerosis, dyslipidemia or other cardiovascular diseases are used in combination with a serine palmitoyltransferase (SPT) inhibitor.

In one embodiment of the present invention, a patient having or at risk of having atherosclerosis can be administered a combination of: 1) serine palmitoyltransferase (SPT) inhibitor; and 2) an additional compound useful to treat atherosclerosis, dyslipidemia, or other cardiovascular diseases, or combinations of compounds useful to treat these diseases.

In addition, a serine palmitoyltransferase (SPT) inhibitor can be administered in combination with other pharmaceutical agents such as cholesterol biosynthesis inhibitors and cholesterol absorption inhibitors, especially HMG-CoA reductase inhibitors and HMG-CoA synthase inhibitors, HMG-CoA reductase and synthase gene expression inhibitors, CETP inhibitors, bile acid sequesterants, fibrates, ACAT inhibitors, squalene synthetase inhibitors, anti-oxidants and niacin. A serine palmitoyltransferase (SPT) inhibitor may also be administered in combination with naturally occurring compounds that act to lower plasma cholesterol levels. These naturally occurring compounds are commonly called nutraceuticals and include, for example, garlic extract, Benecol®, and niacin. A slow-release form of niacin is available and is known as Niaspan. Niacin may also be combined with other therapeutic agents such as lovastatin, which is an HMG-CoA reductase inhibitor and described further below. This combination therapy is known as ADVICOR™ (Kos Pharmaceuticals Inc.).

Any cholesterol absorption inhibitor can be used as the second compound in the combination aspect of the present invention. The term cholesterol absorption inhibition refers to the ability of a compound to prevent cholesterol contained within the lumen of the intestine from entering into the intestinal cells and/or passing from within the intestinal cells into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™ (ezetimibe) (Merck/Schering-Plough).

Any HMG-CoA reductase inhibitor may be employed as an additional compound in the combination therapy aspect of the present invention. The term HMG-CoA reductase inhibitor refers to a compound that inhibits the biotransformation of hydroxymethylglutaryl-coenzyme A to mevalonic acid as catalyzed by the enzyme HMG-CoA reductase. Such inhibition may be determined readily by one of skill in the art according to standard assays (e.g., Methods of Enzymology, 71: 455-509 (1981); and the references cited therein). A variety of these compounds are described and referenced below. U.S. Pat. No. 4,231,938 discloses certain compounds isolated after cultivation of a microorganism belonging to the genus Aspergillus, such as lovastatin. Also, U.S. Pat. No. 4,444,784 discloses synthetic derivatives of the aforementioned compounds, such as simvastatin. Additionally, U.S. Pat. No. 4,739,073 discloses certain substituted indoles, such as fluvastatin. Further, U.S. Pat. No. 4,346,227 discloses ML-236B derivatives, such as pravastatin. In addition, EP 491,226 teaches certain pyridyldihydroxyheptenoic acids, such as rivastatin. Also, U.S. Pat. Nos. 4,681,893 and 5,273,995 disclose certain 6-[2-(substituted-pyrrol-1-yl)-alkyl]-pyran-2-ones such as atorvastatin and the hemicalcium salt thereof (Lipitor®). Other HMG-CoA reductase inhibitors will be known to those skilled in the art, such as rosuvastatin and pitavastatin. Examples of marketed products containing HMG-CoA reductase inhibitors that can be used in combination with compounds of the present invention include Baycol®, Lescol®, Lipitor®, Mevacor®, Pravachol® and Zocor®.

Any HMG-CoA synthase inhibitor may be used as the second compound in the combination therapy aspect of this invention. The term HMG-CoA synthase inhibitor refers to a compound which inhibits the biosynthesis of hydroxymethylglutaryl-coenzyme A from acetyl-coenzyme A and acetoacetyl-coenzyme A, catalyzed by the enzyme HMG-CoA synthase. Such inhibition may be determined readily by one of skill in the art according to standard assays (e.g., Methods of Enzymology, 35:155-160 (1975); and Methods of Enzymology, 110: 19-26 (1985); and the references cited therein). A variety of these compounds are described and referenced below. U.S. Pat. No. 5,120,729 discloses certain beta-lactam derivatives. U.S. Pat. No. 5,064,856 discloses certain spiro-lactone derivatives prepared by culturing the microorganism MF5253. U.S. Pat. No. 4,847,271 discloses certain oxetane compounds such as 11-(3-hydroxymethyl-4-oxo-2-oxetayl)-3,5,7-trimethyl-2,4-undecadienoic acid derivatives. Other HMG-CoA synthase inhibitors will be known to those skilled in the art.

Any compound that decreases HMG-CoA reductase gene expression may be used as an additional compound in the combination therapy aspect of this invention. These agents may be HMG-CoA reductase transcription inhibitors that block the transcription of DNA or translation inhibitors that prevent translation of mRNA coding for HMG-CoA reductase into protein. Such inhibitors may either affect transcription or translation directly, or may be biotransformed into compounds that have the aforementioned attributes by one or more enzymes in the cholesterol biosynthetic cascade or may lead to the accumulation of an isoprene metabolite that has the aforementioned activities. Such regulation is readily determined by those skilled in the art according to standard assays (Methods of Enzymology, 110: 9-19 1985). Several such compounds are described and referenced below however other inhibitors of HMG-CoA reductase gene expression will be known to those skilled in the art. U.S. Pat. No. 5,041,432 discloses certain 15-substituted lanosterol derivatives. Other oxygenated sterols that suppress the biosynthesis of HMG-CoA reductase are discussed by E. I. Mercer (Prog. Lip. Res., 32:357-416 1993).

Any compound having activity as a CETP inhibitor can serve as the second compound in the combination therapy aspect of the instant invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. No. 6,197,786. CETP inhibitors disclosed in these patents include compounds, such as [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot, 49(8): 815-816 (1996), and Bioorg. Med. Chem. Lett.; 6:1951-1954 (1996), respectively.

Any ACAT inhibitor can serve as an additional compound in the combination therapy aspect of this invention. The term ACAT inhibitor refers to a compound that inhibits the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are described and referenced below; however, other ACAT inhibitors will be known to those skilled in the art. U.S. Pat. No. 5,510,379 discloses certain carboxysulfonates, while WO 96/26948 and WO 96/10559 both disclose urea derivatives having ACAT inhibitory activity.

Any compound having activity as a squalene synthetase inhibitor can serve as an additional compound in the combination therapy aspect of the instant invention. The term squalene synthetase inhibitor refers to a compound that inhibits the condensation of two molecules of farnesylpyrophosphate to form squalene, a reaction that is catalyzed by the enzyme squalene synthetase. Such inhibition is readily determined by those skilled in the art according to standard methodology (Methods of Enzymology, 15:393-454 (1969); and Methods of Enzymology, 110: 359-373 (1985); and references cited therein). A summary of squalene synthetase inhibitors has been complied in Curr. Op. Ther. Patents, 861-4, (1993).

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid® and Tricor®. These compounds can also be used in combination with a serine palmitoyltransferase (SPT) inhibitor.

SPT inhibition may be beneficial not only for atherosclerosis, but also for conditions such as type II diabetes, lipotoxicity and insulin sensitivity. It has been shown that chronic exposure to fatty acids due to obesity or hyperglycemia causes apoptosis of pancreatic β-cells (lipotoxicity) and disruption of insulin response via generation of ceramide (M. Shimabukuro et al., Proc Natl Acad Sci USA. 1998;95:2498-502).

Diabetes can be treated by administering to a patient having diabetes (especially Type II), insulin resistance, impaired glucose tolerance, or the like, or any of the diabetic complications such as neuropathy, nephropathy, retinopathy or cataracts, a therapeutically effective amount of a SPT inhibitor in combination with other agents (e.g., insulin) that can be used to treat diabetes. This includes the classes of anti-diabetic agents (and specific agents) described herein.

Any glycogen phosphorylase inhibitor can be used as the second agent in combination with a SPT inhibitor of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a SPT inhibitor of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art.

Any sorbitol dehydrogenase inhibitor can be used in combination with a SPT inhibitor of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Patent Nos. 5,728,704 and 5,866,578 disclose compounds and a method for treating or preventing diabetic complications by inhibiting the enzyme sorbitol dehydrogenase.

Any glucosidase inhibitor can be used in combination with a SPT inhibitor of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C(hydroxymethyl)-D-epi-inositol, and the varioz s N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethy-)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor AI-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used as the second agent in combination with a SPT inhibitor of the present invention, includes, for example, the following: biguanides (e.g., metformin), insulin secretagogues (e.g., sulfonylureas and glinides), glitazones, non-glitazone PPARγ agonists, PPA Rβ agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-β inhibitors and AGE breakers.

As described above, a serine palmitoyltransferase (SPT) inhibitor can be administered alone or with other pharmaceutically active compounds. The other pharmaceutically active compounds can be intended to treat the same disease as the serine palmitoyltransferase (SPT) inhibitor or a different disease. If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup. All combinations, delivery methods and administration sequences are contemplated.

Since one aspect of the present invention contemplates the treatment of the diseases referenced with a combination of pharmaceutically active agents that may be administered separately, the invention further relates to combining separate pharmaceutical compositions in kit form. For example, a kit may comprise two separate pharmaceutical compositions comprising: 1) a serine palmitoyltransferase (SPT) inhibitor; and 2) a second pharmaceutically active compound. The kit also comprises a container for the separate compositions, such as a divided bottle or a divided foil packet. Additional examples of containers include syringes, boxes, bags, and the like. Typically, a kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday, . . . etc. . . . Second Week, Monday, Tuesday,” etc. Other variations of memory aids will be readily apparent. A “daily dose” can be a single tablet or capsule or several pills or capsules to be taken on a given day. Also, a daily dose of a serine palmitoyltransferase (SPT) inhibitor can consist of one tablet or capsule, while a daily dose of the second compound can consist of several tablets or capsules and vice versa. The memory aid should reflect this and assist in correct administration of the active agents.

In another embodiment of the present invention, a dispenser designed to dispense the daily doses one at a time in the order of their intended use is provided. Preferably, the dispenser is equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

All documents cited herein are hereby incorporated by reference.

The examples presented below are intended to illustrate particular embodiments of the invention, and are not intended to limit the scope of the specification, including the claims, in any manner.

Some abbreviations used in this application are defined below:

SM, sphingomyelin;

SPT, serine palmitoyltransferase;

LCAT, lecitine:cholesterol acyltransferase;

LPL, lipoprotein lipase;

PC, plasma phosphatidylcholine;

RT-PCR, real-time polymerase chain reaction;

ApoE, Apolipoprotein E;

WD, Western diet chow-fed ApoE knockout mice;

WD+myr, Western diet chow plus myriocin-fed ApoE knockout mice;

Normal, normal or standard chow-fed ApoE knockout mice;

C57BI/6J, normal or standard chow-fed wild-type control mice;

KO, knockout;

TG, triacylglycerol;

SRE, sterol regulatory elements;

SREBP, sterol regulatory element binding protein;

STD, standard chow;

LC/MS, liquid chromatography/mass spectroscopy

### EXAMPLES

Materials—Cholesterol R1, Triglycerides Reagent and Bovine serum albumin (BSA, fatty acid ultra-free) were purchased from Roche Diagnostics Corporation (Indianapolis, Ind.). Superose 6HR chromatography column was purchased from Pharmacia Biotech (Buckinghamshire, England). Sphinganine, sphingomyelin (brain), 1-palmitoyl-2-oleoyl-sn-glycero-3-phosphocholine, and ceramide were purchased from Avanti Polar Lipids (Alabaster, Ala.). Myriocin, 1,2-hexadecanediol, psychosine, serine, paimitoyl CoA, and Oil Red O were obtained from Sigma (St.Lous, Mo.). IHC Zinc-Tris fixative was purchased from PharMingen (San Diego, Calif.). Normal chow and Western diet chow for rodents were obtained from Research Diet (New Brunswick, N.J.). HPLC grade water, acetonitrile, and butyl alcohol (normal) were from Mallinkrodt (Paris, Ky.). Formic acid (90%) was from Aldrich (Milwaukee, Wis.). Ammonium acetate (F.W. 77.09), trimethylpentane, tetrahydrofuran, acetone, dichloromethane and 2-propanol were obtained from EM Science (Gibbstown, N.J.). Serum amyloid A ELISA kit for mice was obtained from Biosource (Camarillo, Calif.).

Animal experiments—Male C57BI/6J and ApoE KO mice on C57BI/6J background were obtained from the Jackson Laboratory (Bar Harbor, Me.) or Taconic (Germantown, N.Y.) (Plaque formation in ApoE KO mice is lipid-driven (A. S. Plump et al., Cell. 1992;71 (2):343-53)). Myriocin was mixed with Western diet containing 0.21% cholesterol and 21% fat. 8-12-week-old mice received 0.3 mg myriocin/kg/day for 12 weeks (Table 1). 10-12 week-old ApoE KO mice (n=8) were pre-fed a western diet for 2 weeks and mice received various concentrations of myriocin for 4 weeks in diet admix. Control groups consisted of ApoE KO mice fed normal chow or Western diet without myriocin, and normal chow-fed C57BI/6J mice. Body weight and chow feeding was measured every week to examine the food consumption. For the femoral artery cuff model, 8-10 week-old male ApoE KO mice were anethetized and the right femoral artery was dissected from its surroundings. A nonconstrictive polyethylene cuff (Portex, 0.40-mm inner diameter, 0.80-mm outer diameter, and 1.5-mm length) was placed looselyaround the right femoral artery.

TABLE 1
Experimental design

Group

| | 1 | 2 | 3 | 4 |
|---|---|---|---|---|
| N | 16 | 16 | 16 | 16 |
| Strain | ApoE KO | ApoE KO | ApoE KO | C57BI/6J |
| Diet | Western | Western | Standard | Standard |
| Myriocin | 0.3 mg/kg | — | — | — |

SPT expression and activity—Total RNA was isolated from liver and aorta using Trizol (Invitrogen, Calif.). LCB1 and LCB2 mRNA levels were measured by real-time (RT) polymerase chain reaction (PCR) on ABI Prism 7900HT Sequence Detection System (Applied Biosystems, Foster City, Calif.). The following primers and probe sets were utilized: LCB1, forward primer, 5′-CCGCTCCTTCGTGGTTGA-3′: reverse primer, 5′-GAGGTMCGMGCAGAAAAGCAG-3′: probe, 5′FAM-TCAGCGGCTCTCCGGTCAAGGAT-3′; LCB2, forward primer, 5′-CTGGATGAGGCTCACAGCATT-3′, reverse primer, 5′-CCTCAGGATCCAGGCCM-3′, probe, 5′FAM-CCTTCAGGGCGAGGCGTGGTAGAT-3′. The optimum number of cycles was set for each gene product with uniform amplification. Each mRNA level was expressed as a ratio to 18s ribosomal RND or as a ratio to GAPDH RNA.

Liver tissues from each group were homogenized and SPT activity was measured using ¹⁴C-serine and palmitoyl CoA as substrates and thin-layer chromatography (TLC) analysis (K. Gable et al., J. Biol. Chem. 2000;275(11):7597-603).

Analysis of Sphingolipids and Phospholipids by LC/MS and HPLC—Total lipids were extracted by the modified method of Bligh-Dyer extraction (E. G. Bligh and W. J. Dyer, Can. J. Med. Sci. 1959;37:911-917; and D. K. Perry, A. Bielawska and Y. A. Hannun, Methods Enzymol. 2000;312:22-31). A Micromass (Waters Corp., Milford, Mass.) Quattro Ultima tandem quadrupole mass spectrometer with a standard Z-spray™ ion source, set to electrospray positive ionization mode, with MassLynx™ version 3.5 operating software, was used for all quantitative determinations. Source conditions were typically as follows: capillary 3.5 kV, source temperature 110 degrees C., and desolvation temperature 325 degrees C. Multipliers were set to 650 V, and the dwell time for each analyte was 100 milliseconds. Precursor-to-product ion transitions were established through direct infusion of each compound into the mass spectrometer. The following ion transitions were used for quantification: sphingomyelin (704→184 m/z), sphinganine (302→284 m/z), ceramide (566→264 m/z) and psychosine (462→282 m/z) as an internal standard. For the instrument, at a collision cell pressure of 2×10⁻³ mbar argon, cone and collision voltages were as follows: sphingomyelin (45V, 25 eV), sphinganine (45V, 15 eV), ceramide (45V, 25 eV) and psychosine (45V, 25 eV).

The liquid chromatography system was composed of twin Shimadzu (Columbia, Md.) LC-10ADvp HPLC pumps with a SCL-10Avp controller (flow rate 0.2 mL/minute), and a LEAP Technologies (Carrboro, N.C.) CTC PAL autosampler. For the quantitative method, the analytical column was a Phenomenex (Torrance, Calif.) Polar-RP (2.0×150 mm, 4 μm) with a MetaChem (Torrance, Calif.) MetaGuard Polaris C8 2.0 mm direct connect (5 μm) guard column. Mobile phase A consisted of water/acetonitrile/formic acid (60/40/0.1) and mobile phase B was propanol. The HPLC pumps were programmed with a gradient for each injection to deliver 98% mobile phase A (0-1 minute), 30% mobile phase A (1-2 minutes), 30% mobile phase A (2-4 minutes), and 98% mobile phase A (4-4.5 minutes). A sample volume of 2 μL was injected into the LC/MS/MS system. Final chromatographic retention times for sphingomyelin, sphinganine, psychosine (internal standard) and ceramide were 4.84 minutes, 5.43 minutes, 4.92 minutes and 5.31 , minutes, respectively. Lipid extracts were analyzed by HPLC and evaporative light scattering detector to determine plasma sphingomyelin and phosphatidylcholine (PC) levels (R. Homan and M.K. Anderson, J. Chromatogr. B. Biomed. Sci. Appl. 1998;708:21-6).

Plasma lipids and serum amyloid A measurement—Mice were sacrificed by CO₂ inhalation and blood was collected through cardiac puncture. Plasma concentrations of total cholesterol and triglyceride were determined enzymatically on a Cobas Mira Plus auto-analyzer using Cholesterol R1 and Triglycerides Reagent methods, respectively (Roche Diagnostics, Indiana, USA). Colormetric changes were measured at 500 nm. Lipoproteins were separated from mouse plasma by fast-protein liquid chromatography utilizing a Superose 6HR column. Cholesterol distribution among lipoproteins was determined by in-line post column analysis (K. A. Kieft, T. M. Bocan and B. R. Krause, J. Lipid Res. 1991; 32:859-66). Serum Amyloid A (SAA) protein in plasma was measured by ELISA according to the manufacturer's instructions (Biosource).

Vascular pathology—For quantitative analysis of atherosclerotic lesion coverage, sacrificed mice were perfused with saline and the aorta was isolated from the heart to the iliac bifurcation by severing minor branching arteries and dissecting the adventitia. After 24 hrs of fixation with 10% buffered formalin, aorta was opened longitudinally and pinned down on the black wax. Lipids were stained with Oil Red O and photographs were taken. The percentage of aorta stained with Oil Red O was determined by image analysis software, Image Pro Plus.

For histological analysis, the mice were perfused and fixed in Zinc-Tris fixative. Paraffin embedded sections were stained with Masson's Trichrome. Intimal macrophages were immunohistochemically stained using MAC-2 antibody (clone M3/38 from Cedarlane Laboratories Limited) counterstained with Verhoeff elastic stain. T-lymphocytes were immunohistochemically stained with rat CD3 antibody (clone CD3-12, Serotec). Lesion thickness and area occupied by macrophages were determined using Image Pro Plus software.

Statistics—Results are expressed as mean±SEM. The statistical significance of difference between mean values was analyzed using the paired t-test. Comparisons among several groups were determined by one-way ANOVA with Dunnet's post hoc analysis using PRISM 2.01. If a significant difference was found among groups, distribution-free multiple comparisons were performed to find significance among groups. When SEMs were unequal, a nonparametric test (Mann-Whitney) was used to calculate the level of significance. Results were considered significant at P<0.05.

The following Procedures were used in the indicated figures:

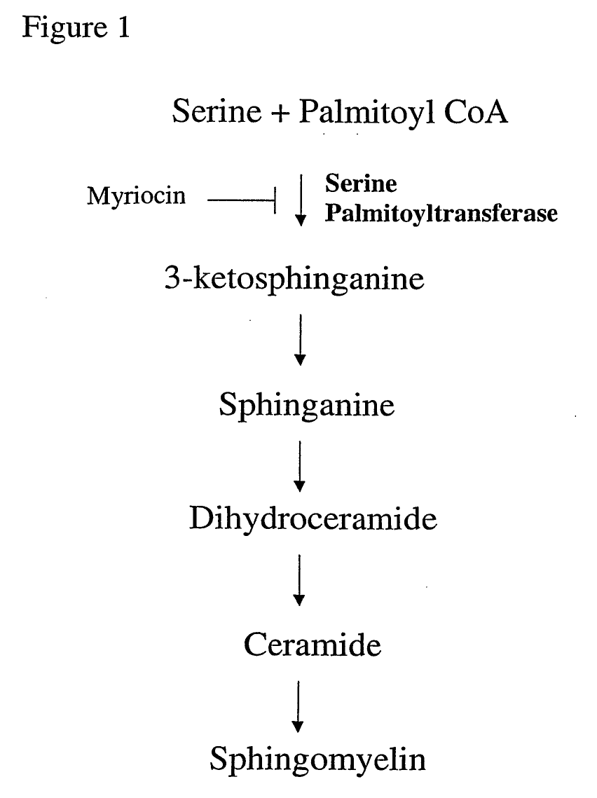

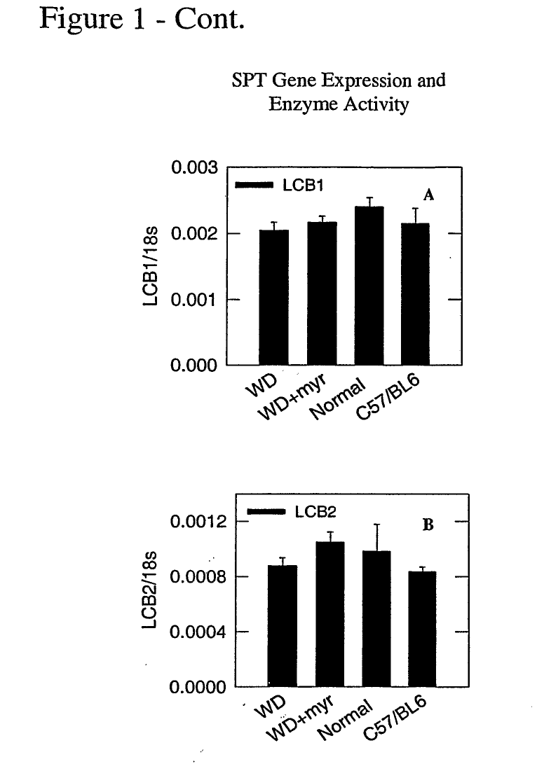

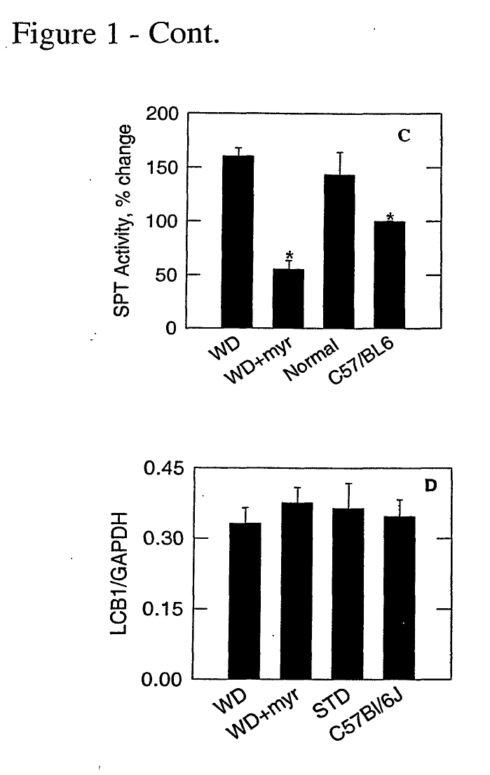

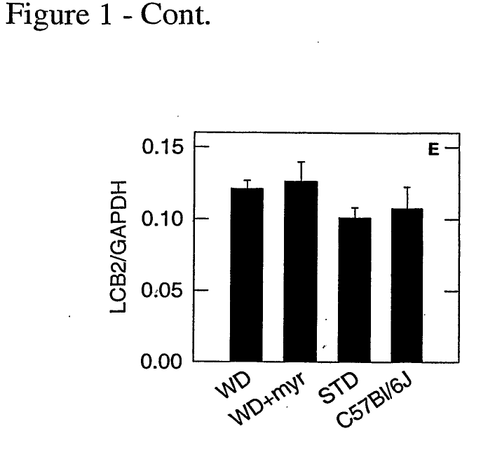

FIG. 1. Sphingomyelin Biosynthetic Pathway. Serine palmitoyltransferase (SPT) is the first rate-limiting step of sphingolipid biosynthesis. Myriocin specifically inhibits SPT reaction.

FIGS. 1-A, 1-B, 1-C, 1-D and 1-E. SPT gene expression and enzyme activity. Mice were treated with 0.3 mg/kg/day myriocin for 12 weeks by mixing with Western diet chow. Liver was isolated and total mRNA and cell-free homogenate were prepared. SPT mRNA expression was quantified by quantitative RT-PCR. Expression was described as a ratio of LCB1 (A) or LCB2 (B) mRNA to 18s RNA or to rodent GAPDH RNA (n=5, P>0.05). SPT activity of cell-free homogenate (C) was measured with ¹⁴C-labeled Serine and palmitoyl-coenzyme A as substrates and analyzed by TLC. Relative amounts of 3-ketosphinganine were determined by densitometry scanning. The values reported are mean±SEM (n=3, *P<0.05).

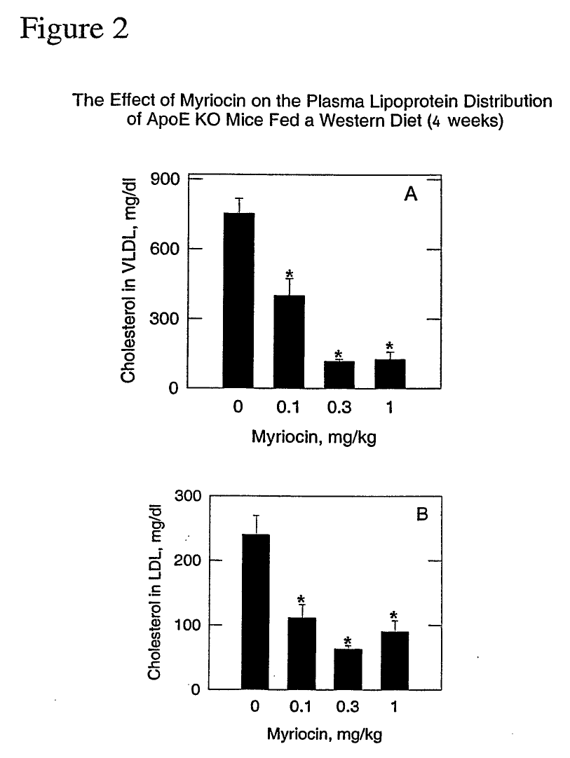

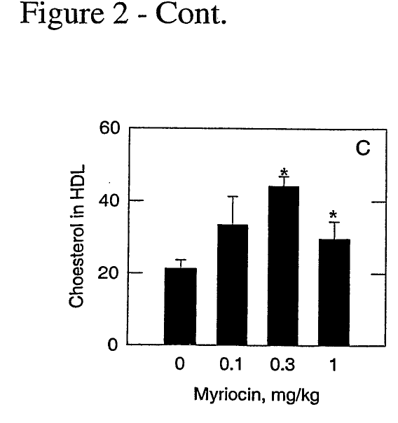

FIG. 2. Plasma Lipoprotein Distribution of ApoE KO Mice Fed a Western Diet. After 4 weeks of diet admix myriocin treatment, mice were sacrificed and plasma was isolated for lipoprotein composition. βVLDL (A), LDL (B) and HDL (C) Lipoproteins were separated from mouse plasma by fast-protein liquid chromatography (FPLC) utilizing a Superose 6HR column. Cholesterol distribution among lipoproteins was determined by in-line post column analysis (Kieft, K. A., T. M. A. Bocan, B. R. Krause, J. Lipid Res. 1991, 32: 859-866). The values reported are mean±SEM (n=8, *P<0.01).

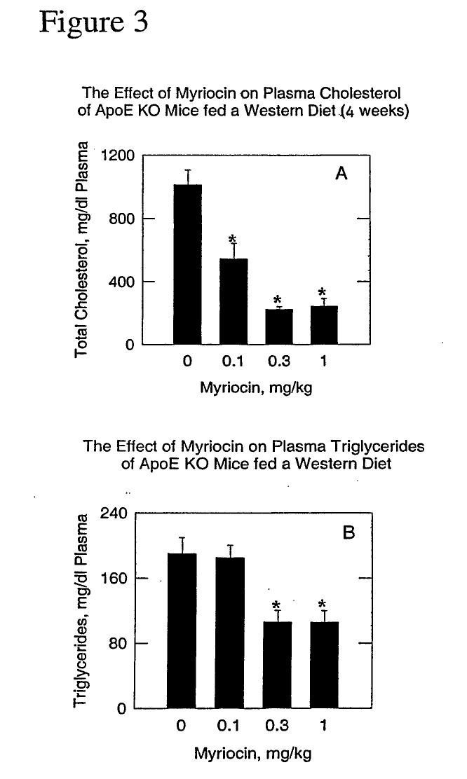

FIG. 3. Plasma Cholesterol and Triglycerides in ApoE KO Mice Fed a Western Diet. After 4 weeks of diet admix myriocin treatment, mice were sacrificed and plasma was isolated. Plasma concentrations of total cholesterol (A) and triglyceride (B) were determined enzymatically on a Cobas Mira Plus auto-analyzer using Cholesterol R1 and Triglycerides Reagent methods, respectively. Colormetric changes were measured at 500 nm. The values reported are mean±SEM (n=8, *P<0.01).

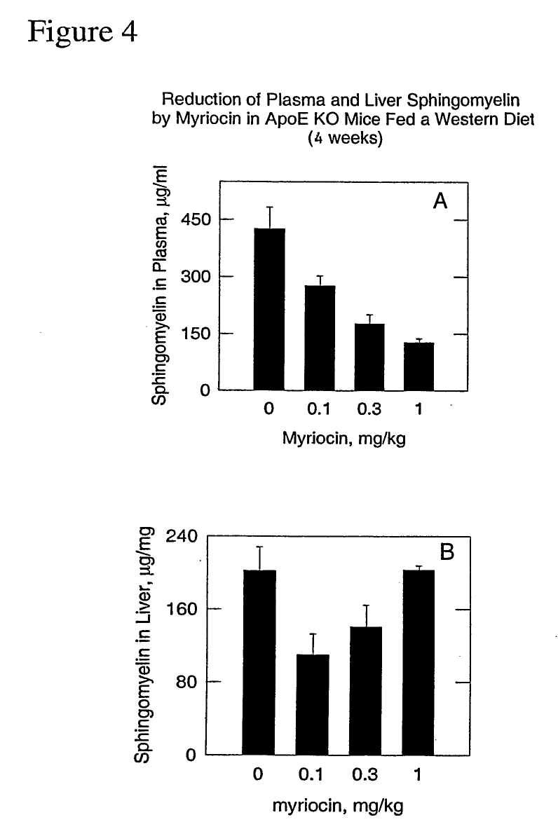

FIG. 4. Plasma and Liver Sphingomyelin in ApoE KO Mice Fed a Western Diet. After 4 weeks of diet admix myriocin treatment, mice were sacrificed and plasma and liver were isolated. Total lipids were extracted by Chloroform:Methanol:Water (1:1:0.9) and followed by phase separation. Sphingomyelin levels in plasma (A), and liver (B) were determined by LC/MS. The values reported are mean±SEM (n=5, P<0.05).

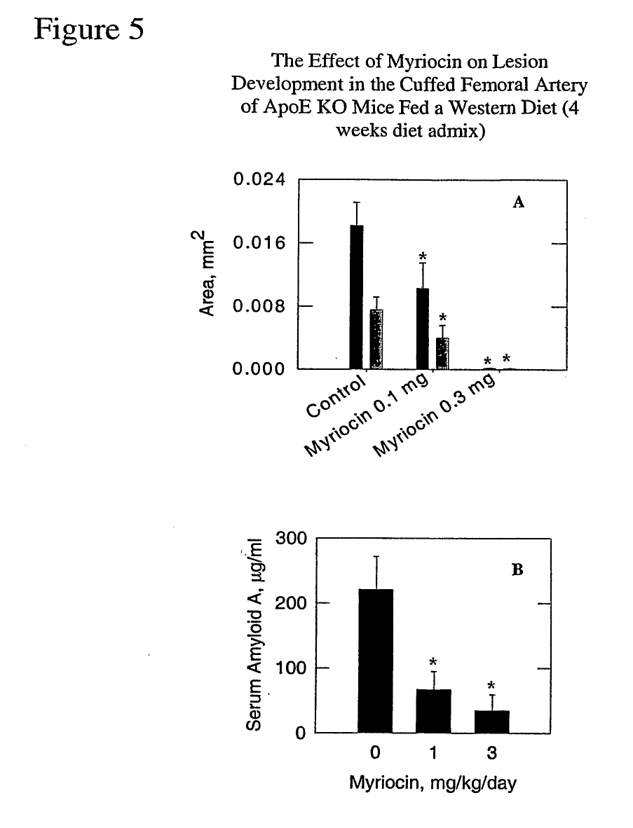

FIG. 5. Lesion Development in the Cuffed Femoral Artery of ApoE KO mice Fed a Western Diet. Mice were anesthetized and the right femoral artery was dissected from its surroundings. A nonconstrictive polyethylene cuff (Portex, 0.40-mm inner diameter, 0.80-mm outer diameter, and 1.5-mm length) was placed looselyaround the right femoral artery. Cuffed ApoE KO mice were fed a western diet mixed with myriocin at various concentrations for 4 weeks. Mice were sacrificed and femoral artery were isolated and embedded in paraffin. Cross-sections of femoral artery were stained with Masson's Trichrome or Mac II antibody. Atherosclerotic lesion (black bar) and macrophage size (gray bar) in the femoral artery were quantified by using Image Pro Plus software (FIG. 5A). Plasma serum amyloid A levels were determined by colormetric ELISA (FIG. 5B). The value reported were the means±SEM (n=6-8, *P<0.05). Bar represents 100 pm.

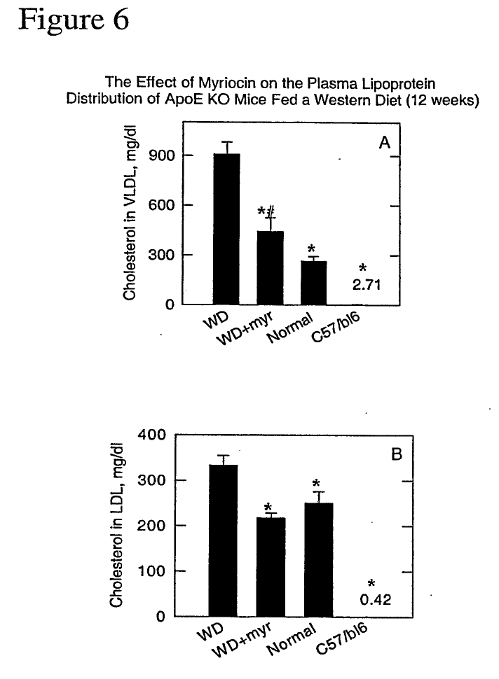

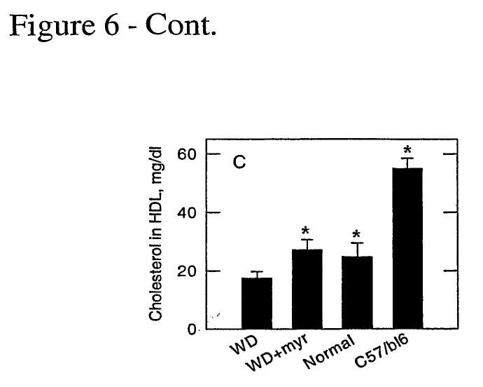

FIG. 6. Plasma Lipoprotein Distribution of ApoE KO Mice Fed a Western Diet. After 12 weeks of diet admix myriocin treatment (0.3 mg/kg/day), mice were sacrificed and plasma was isolated for lipoprotein composition. βVLDL (A), LDL (B) and HDL (C) Lipoproteins were separated from mouse plasma by fast-protein liquid chromatography (FPLC) utilizing a Superose 6HR column. Cholesterol distribution among lipoproteins was determined by in-line post column analysis (Kieft, K. A., T. M. A. Bocan, B. R. Krause, J. Lipid Res. 1991, 32:859-866.). The values reported are mean±SEM (n=5, *P<0.01, Western diet vs other study groups; n=5, #P<0.05, Western diet plus myriocin vs normal chow).

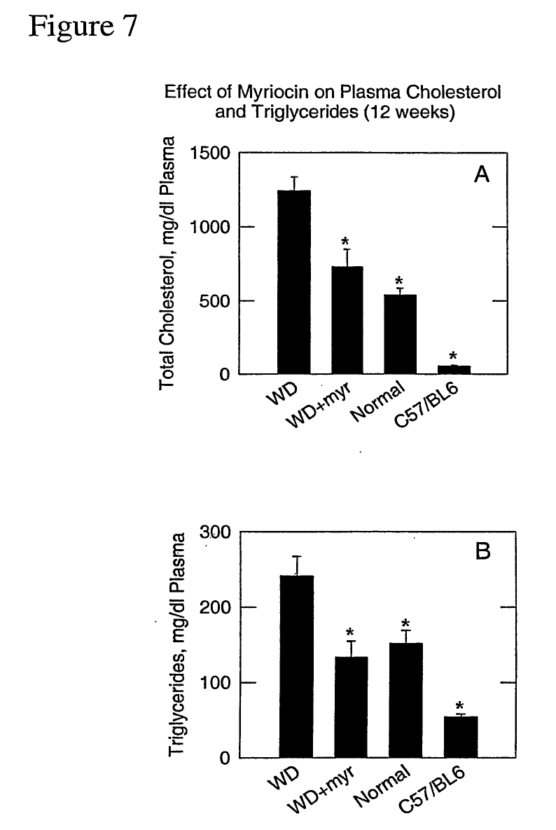

FIG. 7. Cholesterol and Triglyceride concentrations in plasma. After 12 weeks of myriocin treatment, mice were sacrificed and plasma was isolated. Plasma concentrations of total cholesterol (A) and triglyceride (B) were determined enzymatically on a Cobas Mira Plus auto-analyzer using Cholesterol R1 and Triglycerides Reagent methods, respectively. Colormetric changes were measured at 500 nm. The values reported are mean±SEM (n=5, *P<0.01).

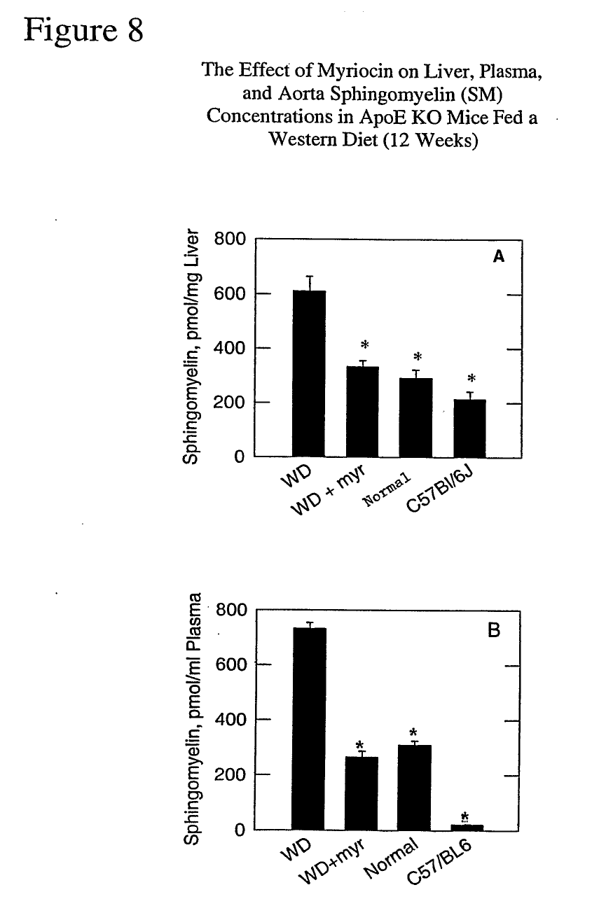

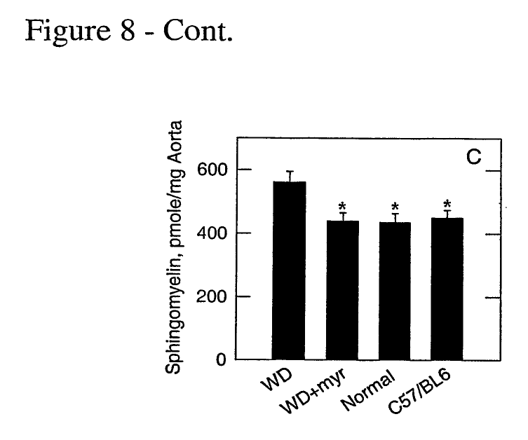

FIG. 8. Sphingomyelin concentrations in liver, plasma and aorta. After 12 weeks of myriocin treatment, mice were sacrificed and plasma, liver and aorta were isolated. Total lipids were extracted by a modified Blier-Dyer method. Sphingomyelin levels in liver (A), plasma (B), and aorta (C) were determined by LC/MS. The values reported are mean±SEM (n=5, *P<0.05).

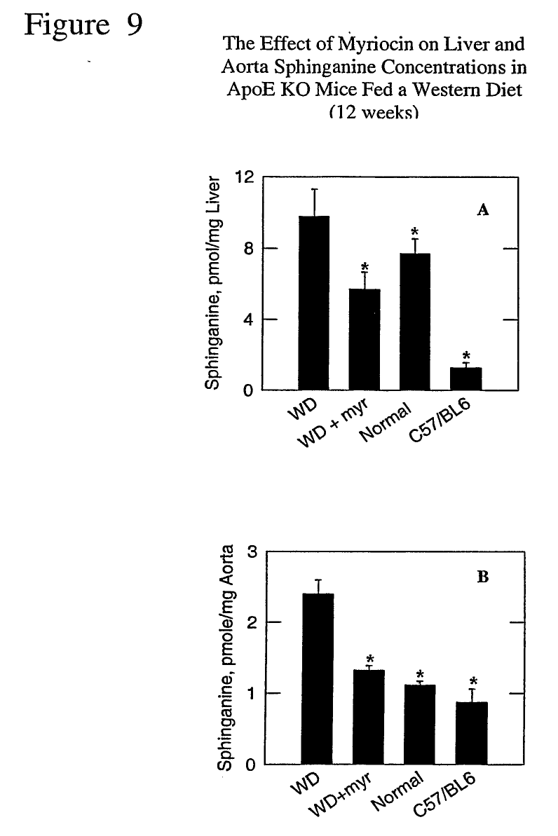

FIG. 9. Sphinganine concentrations in liver and aorta. After 12 weeks of myriocin treatment, mice were sacrificed and plasma, liver and aorta were isolated. Total lipids were extracted by Blier-Dyer method. Sphinganine levels in liver (A) and aorta (B) were determined by LC/MS. The values reported are mean±SEM (n=5, *P<0.05).

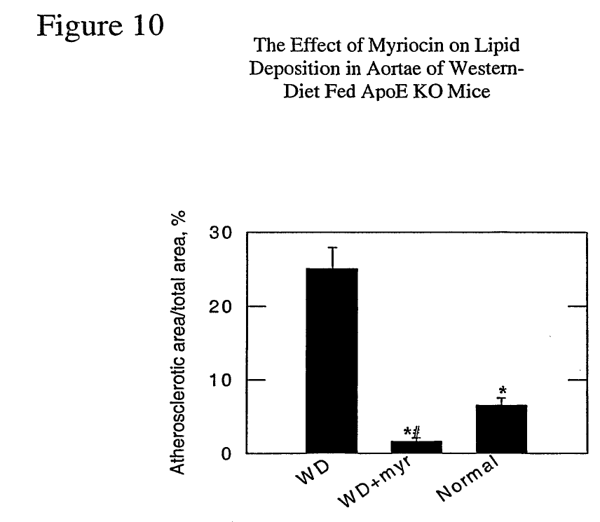

FIG. 10. Lipid deposition in aortae of Western-diet fed ApoE KO mice. ApoE KO mice were fed with Western diet in the presence or absence of myriocin for 12 weeks. Mice were sacrificed and fixed with 10% buffered formalin for 24 hours. The aorta from heart to the iliac bifurcation was dissected, opened along the ventral surface and pinned down on a black wax background. Accumulated lipids were visualized by Oil Red O staining. Areas of the atherosclerotic lesion were quantified by using Image Pro Plus and represented as percentage of lesion area to total aorta area. The values reported are mean±SEM (n=4, WD vs. WD+myriocin or normal chow, *P<0.01; Normal vs. WD+myriocin, #P<0.01). Bar represents 1 cm.

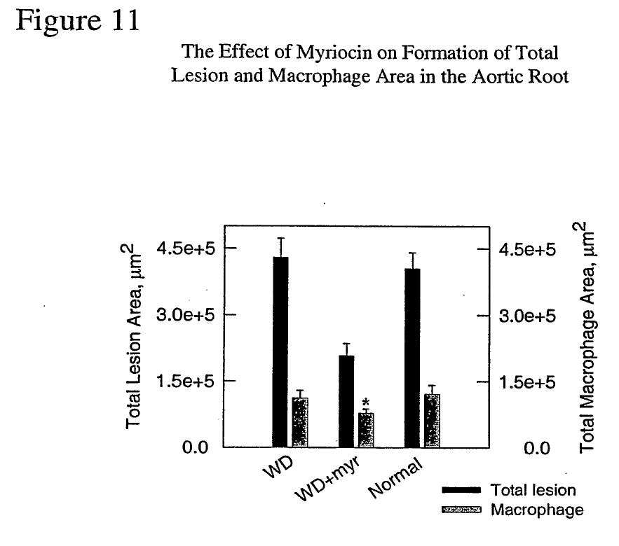

FIGS. 11 & 12. Formation of atherosclerotic lesions in aortic root and brachiocephalic artery. ApoE KO mice fed with Western-diet in the absence or presence of myriocin, ApoE KO mice and C57BI/6J mice fed with normal chow were sacrificed and fixed with Zinc-Tris. The cross-section of brachiocephalic artery was stained by Masson's Trichrome and MAC-2 antibody counterstained with Verhoeff elastic stain. Atherosclerotic lesion (black bars) and macrophage size (gray bars) in brachiocephalic artery and in aortic root were quantified by using Image Pro Plus. The values reported are mean±SEM (n=5, *P<0.01, WD vs WD plus myriocin; #P<0.01, normal vs WD plus myriocin). Bar represents 100 μm.

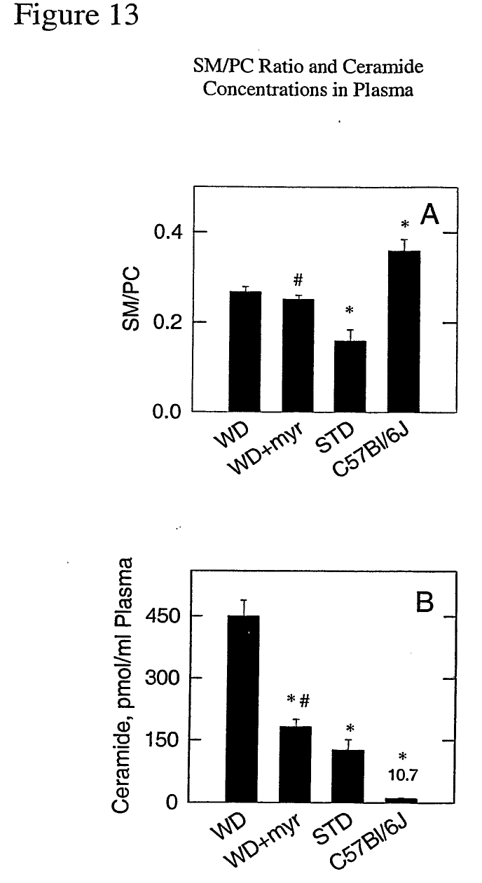

FIG. 13. SM/PC ratio and ceramide concentrations in plasma. Plasma concentrations of SM and PC were determined, and SM/PC ratio (FIG. 13A) was calculated using HPLC. Plasma ceramide levels (FIG. 13B) were analyzed by LC/MS/MS. Values are mean±SEM (n=5; *P<0.01, Western diet vs Western diet plus myriocin; #P<0.05, standard chow vs Western diet plus myriocin).

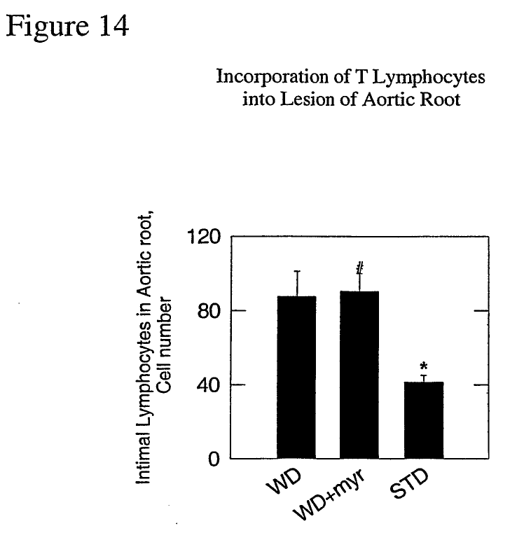

FIG. 14. Incorporation of T lymphocytes into lesion of aortic root. Cross section of aortic root was stained by rat CD3 antibody and developed by diaminobenzidine (brown color) to detect incorporated T lymphocytes. Sections were counterstained with Harris hematoxylin (blue). T-lymphocyte incorporation was quantified by measuring number of intimal T lymphocytes in aortic root (FIG. 14). Values are mean±SEM (n=5; *P<0.05, Western diet vs standard chow; #P<0.05, Western diet plus myriocin vs standard chow). Bar represents 50 μm.

The following Results were obtained as indicated in the referenced Figures:

SPT gene expression and enzyme activity—RT-PCR analysis demonstrated that myriocin had no effect on expression of LCB1 and LCB2 mRNA (FIG. 1A, B, D and E) in the liver. Compared with C57BI/6J mice, SPT activity was increased in ApoE KO mice fed a Western diet and normal chow for 12 weeks by 60% (n=3, P<0.05) and 43% (n=3, P<0.05), respectively (FIG. 1C). Myriocin dramatically lowered SPT activity in the liver of the Western diet-fed ApoE KO mice (66% decrease compared with the untreated Western diet-fed ApoE KO mice and 48% compared with the C57BI/6J control group (n=3, P<0.05)). Thus, myriocin treatment had no effect on SPT expression, but was extremely effective in lowering SPT enzyme activity in the liver.

Lipid composition—Myriocin treatment significantly lowered plasma levels of cholesterol and TG in a dose-dependent manner (FIG. 3). Cholesterol levels in plasma were significantly affected by inhibition of sphingolipid biosynthesis. At 0.1 mg myriocin/kg/day, plasma cholesterol was reduced by 46% compared to no-myriocin control and it reached a maximum of 76% decrease at 0.3 mg myriocin/kg/day dose (FIG. 3A). Compared to cholesterol, the degree of TG lowering effect by myriocin was smaller. Although there was no effect of myriocin on plasma TG levels at 0.1 mg/kg/day, plasma TG levels were lowered at 0.3 mg/kg/day by 44% (FIG. 3B). In addition, myriocin lowered VLDL- and LDL-cholesterol dramatically by 83% and 63% at maximum, respectively (FIG. 2A, B). In contrast, HDL-cholesterol was raised by 2.1-fold by inhibition of SM synthesis (FIG. 2C). Therefore, plasma lipid profile was significantly influenced by myriocin, an SPT inhibitor.

To examine the effect of myriocin on sphingolipid biosynthesis, sphingomyelin (SM) levels in plasma and liver were measured by LC/MS. In plasma, 1 mg/kg/day myriocin treatment lowered the SM levels by 70% (FIG. 4A). In contrast, SM levels in the liver were decreased to a maximum of 46% at 0.1 mg/kg/day myriocin. In liver, at 1 mg/kg/day, SM levels were comparable to the non-treated group (FIG. 4B). Therefore, SPT inhibition for 4 weeks lowered the overall lipid levels in plasma and liver in a dose-dependent manner.

To investigate whether myriocin was effective in lowering sphingomyelin biosynthesis, various concentrations of myriocin were administered to ApoE KO mice for 4 weeks and plasma SM levels were examined. HPLC analysis of plasma lipids demonstrated that myriocin dramatically lowered plasma SM levels in a dose-dependent manner (Table 2). At the highest dose of myriocin (1 mg/kg/day), plasma sphingomyelin levels were reduced by 70% when compared with no-myriocin control. Thus, myriocin in a diet admix was effective in inhibiting sphingomyelin biosynthesis and lowering plasma sphingomyelin levels. Since SM levels relative to phospholipids has been regarded as a risk factor for coronary artery disease, plasma PC levels were determined by HPLC analysis. Although there were significant changes in plasma SM levels, plasma PC levels were not changed to the same extent by myriocin. Consequently, SM/PC molar ratio was lowered by myriocin in a dose-dependent manner (Table 2). Thus, myriocin exerted profound SM-lowering effect without affecting PC biosynthesis or degradation significantly.

TABLE 2
Plasma sphingomyelin (SM), phosphatidylcholine (PC)
levels in plasma of ApoE knockout mice.

| | | | SM; PC |
|---|---|---|---|
| | SM (nmol/ml) | PC (nmol/ml) | molar ratio |
| WD | 0.584 ± 0.077 | 2.651 ± 0.210 | 0.220 ± 0.027 |
| WD + 0.1 mg myr^{α} | 0.379 ± 0.035 | 2.763 ± 0.195 | 0.137 ± 0.005^{b} |
| WD + 0.3 mg myr^{α} | 0.243 ± 0.032 | 1.999 ± 0.085 | 0.122 ± 0.013^{b} |
| WD + 1 mg myr^{α} | 0.175 ± 0.013 | 2.044 ± 0.146 | 0.086 ± 0.008^{b} |

WD, western diet fed ApoE KO mice;
myr, myriocin.
^{a}myriocin was administered by diet admix for 4 weeks
^{b}P < 0.05, n = 10, vs. WD group

Atherogenesis in the cuffed femoral artery—To determine the lipid lowering effect of myriocin on atherogenesis, the femoral artery of ApoE KO mice were cuffed using a nonconstrictive polyethylene cuff. In addition to the high lipid containing diet (western diet), the cuffing of artery accelerates the development of atherosclerosis. After 4 weeks of western diet, the cuffed femoral artery of ApoE KO mice developed the atherosclerotic-like lesions to a near-total occlusion of lumen mainly composed of macrophage (FIG. 5). In contrast, myriocin treatment (0.1 mg/kg/day) reduced the development of atherosclerotic lesions and macrophage accumulation by 43% and 47%, respectively (FIG. 5A). At 0.3 mg myriocin/kg/day dose, the lesion area was reduced by more than 98% when compared to no-myriocin control (FIG. 5A). Plasma SAA levels which reflect the involvement of inflammatory response were also measured. Myriocin treatment lowered plasma SM by 84% (FIG. 5B). Thus, myriocin reduces atherogenesis of the cuffed femoral artery of ApoE KO mice via lipid-lowering effect and reduction of inflammatory protein levels.

Plasma cholesterol and triglycerides—To determine the effect of myriocin on lipoprotein metabolism, the cholesterol profile in lipoproteins was examined using isolated plasma by FPLC (fast performance liquid chromatography). Myriocin treatment lowered the βVLDL- and LDL-cholesterol by 51% and 35%, respectively (FIG. 6A, B), when compared with Western diet-fed ApoE KO mice. In contrast, HDL-cholesterol content was increased by 54% (FIG. 6C). Cholesterol distribution in lipoproteins of standard chow-fed ApoE KO mice were comparable to those of myriocin-treated ApoE KO mice. Compared to ApoE KO mice, the WT C57BI/6J mice showed very low total cholesterol in plasma. Most of cholesterol content in C57BI/6J mice was found in HDL (55.3 mg/dl in total plasma cholesterol, 58.4 mg/dl). In addition, myriocin lowered plasma apoB levels, which were comparable to standard chow-fed group. As plasma apoB levels, especially apoB100 levels, in LDL correlate with atherogenesis (K. Skalen et al., Nature. 2002;417:750-4), the apoB-lowering effect may contribute to prevention of atherogenesis by myriocin. Thus, the inhibition of sphingolipid biosynthesis has a significant effect on cholesterol distribution in lipoproteins in plasma.

Since SM content of lipoproteins affects the activities of enzymes involved in lipid metabolism in vitro, it was questioned whether the inhibition of sphingolipid biosynthesis affected total cholesterol and triglyceride (TG) levels in plasma. Plasma cholesterol (FIG. 7A) and TG (FIG. 7B) were the highest in Western diet-fed ApoE KO mice and the lowest in control C57BI/6J mice with standard chow-fed ApoE KO mice situated in between. Myriocin exhibited significant lipid-lowering activity by bringing both parameters to the levels of standard chow-fed ApoE KO mice. Myriocin lowered plasma cholesterol and TG by 41% and 45%, respectively (FIG. 7). Therefore, it appears that myriocin lowered the overall lipid levels by affecting enzyme activities involved in lipid metabolism.

Sphingolipid biosynthesis—Although SM levels are determined by both synthesis and degradation, in our experimental system, SM changes were generally associated with changes in SPT activity and sphinganine production, thereby emphasizing the role of the SPT dependent synthetic pathway. Specifically, SM levels in the liver of C57BI/6J mice were significantly lower than those in Western diet-fed ApoE KO mice (myriocin-treated and standard chow-fed alike). Myriocin treatment lowered SM accumulation in liver significantly compared to Western diet-fed ApoE KO mice (FIG. 8A).

Moreover, Western diet-fed ApoE KO mice displayed the highest level of plasma SM, 33 times higher than C57BI/6J and more than two times higher than standard chow-fed ApoE KO mice (FIG. 8B). Myriocin treatment lowered plasma SM in Western diet-fed ApoE KO mice by 64% bringing it to the level of their standard chow-fed counterpart.

Small differences were observed among aortas of various treatments. However, there were statistically significant differences between Western diet-fed ApoE KO and control C57BI/6J mice. Myriocin decreased SM levels by 20% (FIG. 8C). Thus, SPT inhibition by myriocin drastically affected SM production and accumulation in the liver, plasma and aorta.

Certain SM features may determine its fate and potential role in atherosclerosis. Secretory SMase is known to cause SM hydrolysis to generate ceramide, which stimulates aggregation of lipoproteins and foam cell formation (S. L. Schissel et al., J. Biol. Chem. 1998;273:2738-46). High SM/PC ratio in lipoproteins determines their susceptibility to SMase. Plasma SM/PC ratio was measured using HPLC. Although, compared to the Western diet-fed group, plasma SM and PC levels were substantially lower in the myriocin-treated group, myriocin did not affect the plasma SM/PC ratio (FIG. 13A). On the other hand, myriocin treatment reduced ceramide levels by 60%, which is comparable to standard chow-fed group (FIG. 13B). The lowest ceramide levels were found in C57BI/6J control group. Thus, myriocin-induced reduction of SM accumulation was accompanied by substantial reduction in ceramide levels and was not associated with any changes in the SM/PC ratio.

SM synthesis, accumulation and characteristics—To determine if inhibition of SPT activity was translated into an inhibition of SM production, the quantity of sphinganine, an intermediate of SM synthesis (FIG. 1) and a close down-stream marker of SPT activity that cannot be influenced by SM degradation via Smase, was measured. In the liver, sphinganine levels were significantly increased in Western diet-fed as well as standard chow-fed ApoE KO mice compared to control C57BI/6J mice indicating increased rate of SM synthesis in this model of atherosclerosis. Myriocin treatment lowered sphinganine levels in the liver by 42% compared to the Western diet-fed ApoE KO mice (FIG. 9A).

In aorta, sphinganine levels in the myriocin-treated ApoE KO mice, standard chow-fed ApoE KO mice, and control C57BI/6J mice were lower by 45%, 54%, and 63%, respectively, compared to Western diet-fed group (FIG. 9B). Sphinganine in plasma was below detectable levels. Thus, myriocin treatment inhibited the SM synthetic pathway in both the liver and aorta.

Atherosclerosis—Oil Red O staining of en face aortas reveled that myriocin treatment reduced atherosclerotic lesion coverage in Western diet-fed ApoE KO mice by 93%. When compared with normal chow-fed ApoE KO mice, the atherosclerotic lesion of myriocin-treated ApoE KO mice was decreased by 75% (FIG. 10).

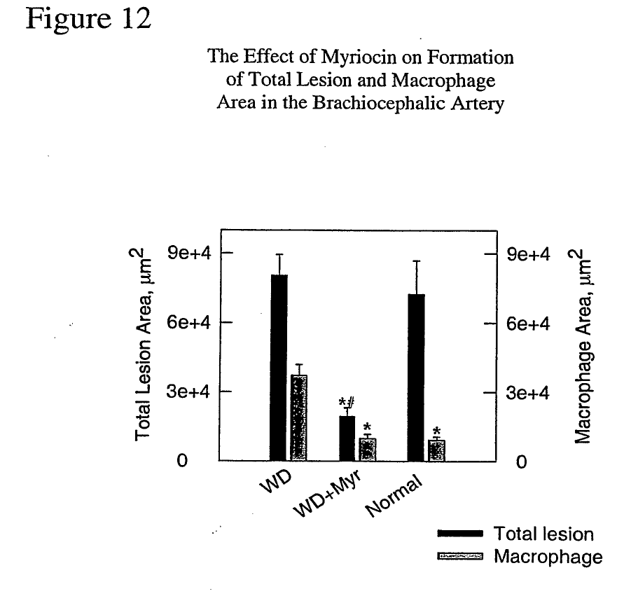

In addition, growth of atherosclerotic lesions in the brachiocephalic artery and aortic valve area were also significantly inhibited (FIGS. 11 and 12). In the aortic root, the lesion area was decreased by 44% and the macrophage area decreased by 31% by myriocin treatment. In contrast, no change was observed between the Western diet-fed and normal chow-fed ApoE KO mice (FIG. 11).

In the brachiocephalic artery, myriocin treatment leads to 76% decrease in lesion area and 74% decrease in macrophage area (FIG. 12). Of note, lesions in myriocin-treated, Western diet-fed ApoE KO mice did not develop necrotic core. Accumulation of T cells was not affected by myriocin treatment (FIG. 14). Thus, SPT inhibition had substantial lipid-lowering and anti-atherogenic effects.

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention.

1-9. (canceled)

10. A pharmaceutical composition comprising:
a) a compound that is a serine palmitoyltransferase inhibitor; and
b) a second compound useful for the treatment of atherosclerosis or dyslipidemia.

11-15. (canceled)

16. A method of lowering plasma lipids comprising administering a therapeutically effective amount of a serine palmitoyltransferase inhibitor to a mammal in need thereof.

17. A method for elevating high density lipoprotein particles comprising administering a therapeutically effective amount of a serine palmitoyltransferase inhibitor to a mammal in need thereof.

18. A method for lowering very low density lipoprotein particles and low density lipoprotein particles comprising administering a therapeutically effective amount of a serine palmitoyltransferase inhibitor to a mammal in need thereof.

19. A method for lowering plasma triglyercides particles comprising administering a therapeutically effective amount of a serine palmitoyltransferase inhibitor to a mammal in need thereof.

20. A method for lowering serum levels of total cholesterol comprising administering a therapeutically effective amount of a serine palmitoyltransferase inhibitor to a mammal in need thereof.

21. A method for improving plasma lipid profile comprising administering a therapeutically effective amount of a serine palmitoyltransferase inhibitor to a mammal in need thereof.

22. A method for inhibiting plaque formation comprising administering a therapeutically effective amount of a serine palmitoyltransferase inhibitor to a mammal in need thereof.

23. A method of reducing the size of plaque comprising administering a therapeutically effective amount of a serine palmitoyltransferase inhibitor to a mammal in need thereof.

24. A method of reducing the size of an atherosclerotic lesion comprising administering a therapeutically effective amount of a serine palmitoyltransferase inhibitor to a mammal in need thereof.

25. A method of reducing the size of a macrophage foam cell comprising administering a therapeutically effective amount of a serine palmitoyltransferase inhibitor to a mammal in need thereof.

26. A method for preventing plaque rupture comprising administering a therapeutically effective amount of a serine palmitoyltransferase inhibitor to a mammal in need thereof.

27. A method for treating dyslipidemia which comprises administering a therapeutically effective amount of a serine palmitoyltransferase inhibitor to a mammal in need thereof.

28. A method for treating atherosclerosis which comprises administering a therapeutically effective amount of a serine palmitoyltransferase inhibitor to a mammal in need thereof.

29. A method for treating diabetes which comprises administering a therapeutically effective amount of a serine palmitoyltransferase inhibitor to a mammal in need thereof.

30. A method for treating metabolic syndrome which comprises administering a therapeutically effective amount of a serine palmitoyltransferase inhibitor to a mammal in need thereof.

31. The method as recited in claims 16-31, in which the serine palmitoyltransferase inhibitor is myriocin.

32. The composition of claim 10 wherein the second compound is an HMG-CoA reductase inhibitor, an HMG-CoA synthase inhibitor, an HMG-CoA reductase gene expression inhibitor, an HMG-CoA synthase gene expression inhibitor, a CETP inhibitor, a bile acid sequestrant, a cholesterol absorption inhibitor, a cholesterol biosynthesis inhibitor, a squalene synthetase inhibitor, a fibrate, niacin, a combination of niacin and lovastatin or an antioxidant.

33. The composition of claim 32 wherein the second compound is an HMG-CoA reductase inhibitor.

34. The composition of claim 33 wherein the second compound is lovastatin, simvastatin, pravastatin, fluvastatin, atorvastatin, rivastatin, rosuvastatin or pitavastatin.

35. The composition of claim 32 wherein the second compound is a CETP inhibitor.

36. The composition of claim 10 wherein the serine palmitoyltransferase inhibitor is myriocin.

37. A kit that comprises:
a) a serine palmitoyltransferase inhibitor and a pharmaceutically acceptable carrier, vehicle or diluent in a first unit dosage form;
b) a second compound that is useful for the treatment of atherosclerosis or dyslipidemia and a pharmaceutically acceptable carrier, vehicle or diluent in a second unit dosage form; and
c) a means for containing the first and second unit dosage forms.

38. The kit of claim 37 wherein the second compound is an HMG-CoA reductase inhibitor, an HMG-CoA synthase inhibitor, an HMG-CoA reductase gene expression inhibitor, an HMG-COA synthase gene expression inhibitor, a CETP inhibitor, a bile acid sequestrant, a cholesterol absorption inhibitor, a cholesterol biosynthesis inhibitor, a squalene synthetase inhibitor, a fibrate, niacin, a combination of niacin and lovastatin or an antioxidant.

39. The kit of claim 38 wherein the second compound is an HMG-CoA reductase inhibitor.

40. The kit of claim 39 wherein the second compound is lovastatin, simvastatin, pravastatin, fluvastatin, atorvastatin, rivastatin, rosuvastatin or pitavastatin.

41. The kit of claim 38 wherein the second compound is a CETP inhibitor.

41. The kit of claim 37 wherein the serine palmitoyltransferase inhibitor is myriocin.
